(19) United States
(12) Patent Application Publication
(10) Pub. No.: US 2026/0091220 A1
(43) Pub. Date: Apr. 2, 2026

(54) TRANSDUCER ARRAYS FOR A SUBJECT'S BODY FOR TUMOR TREATING FIELDS TREATMENT

(71) Applicant: Novocure GmbH, Baar (CH)

(72) Inventors: Elie YAACOBI, Haifa (IL); Noa HALAVEE, Tel Aviv (IL); Stas OBUCHOVSKY, Haifa (IL); Golan BAR-TAL, Haifa (IL); Amir ARIEL, Haifa (IL)

(73) Assignee: Novocure GmbH, Baar (CH)

(21) Appl. No.: 19/343,838

(22) Filed: Sep. 29, 2025

Related U.S. Application Data
(60) Provisional application No. 63/701,217, filed on Sep. 30, 2024.
(60) Provisional application No. 63/836,085, filed on Jun. 30, 2025.

(51) Int. Cl. A61N 1/04; A61N 1/40

(52) U.S. Cl. CPC A61N 1/0476; A61N 1/0408; A61N 1/0496; A61N 1/40

(57) ABSTRACT

A transducer apparatus for delivering tumor treating fields to a subject's body comprises: a plurality of electrode subassemblies configured to be positioned over the subject's body with a front face facing the subject's body and a back face opposite the front face, each electrode subassembly comprising at least one electrode element; a plurality of sections of an anisotropic material layer, each electrically coupled to at least one electrode element, each section of anisotropic material layer comprising a front face and a back face opposite the front face, and wherein when viewed from a direction perpendicular to the front face of the electrode subassemblies, each respective section of anisotropic material layer is spaced apart and is not touching other sections of the plurality of sections of anisotropic material layer; and a flexible electrical connector electrically connecting the plurality of electrode subassemblies.

### CROSS-REFERENCE TO RELATED APPLICATIONS

This application claims priority to U.S. Provisional Patent Application No. 63/836,085, filed Jun. 30, 2025, and U.S. Provisional Ser. No. 63/701,217, filed Sep. 30, 2024, both of which are incorporated herein by reference in their entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. In current commercial systems, TTFields are induced non-invasively into a region of interest by transducer apparatuses (also known as transducer arrays, transducers, electrode arrays, or electrode assemblies) placed on the patient's body and applying alternating current (AC) voltages between the transducers. Conventionally, one or more pairs of transducer apparatuses (e.g., a first pair of transducer apparatuses and a second pair of transducer apparatuses) are placed on the subject's body. AC voltage is applied between the first pair of transducer apparatuses for a first interval of time to generate an electric field with field lines generally running in the front-back direction. Then, AC voltage is applied at the same frequency between the second pair of transducer apparatuses for a second interval of time to generate an electric field with field lines generally running in the right-left direction. The system then repeats this two-step sequence throughout the treatment.

### BRIEF DESCRIPTION OF THE FIGURES

FIGS. 1A-1H depict cross-sectional views of exemplary transducer apparatuses.

FIGS. 2A-2B depict exemplary arrangements of substrate and segmented anisotropic material layers for use in a transducer apparatus.

FIGS. 3A-3K depict exemplary arrangements of segmented anisotropic material layers for use in a transducer apparatus.

FIGS. 4A-4B depict exemplary arrangements of segmented anisotropic material layers for use in a transducer apparatus, wherein the segments are connected by thin bridges.

FIGS. 5A-5D depict exemplary arrangements of segmented anisotropic material layers for use in a transducer apparatus.

FIGS. 6A-6H depict exemplary arrangements of segmented anisotropic material layers for use in a transducer apparatus.

FIG. 7 depicts a transducer apparatus having an exemplary arrangement of anisotropic material layer segments.

FIGS. 8A-8B depicts exemplary transducer apparatuses having exemplary arrangements of anisotropic material layer segments with flexible electrical connectors connected thereto.

FIGS. 9A-9E depict exemplary arrangements of segmented anisotropic material layers for use in a transducer apparatus, where the segmented anisotropic material layers have the same shape and are rotationally symmetric.

FIGS. 10A-10B depict exemplary arrangements of segmented anisotropic material layers for use in a transducer apparatus, where the segmented anisotropic material layers have the same shape.

FIGS. 11A-11B depict exemplary arrangements of segmented anisotropic material layers with segmented substrates for use in a transducer apparatus.

FIGS. 12A-12E depict top plan views of exemplary transducer apparatuses which include an electrode superassembly.

FIGS. 13A and 13B depict a top plan view (i.e., a back face plan view) and a bottom view (i.e., a front face view), respectively, of an example transducer apparatus according to some embodiments.

FIGS. 14A and 14B depict perspective views of example transducer apparatuses on a mannequin according to some embodiments.

Various embodiments are described in detail below with reference to the accompanying drawings, wherein like reference numerals represent like elements, and wherein descriptions of like elements may not be repeated for every embodiment, but may be considered to be the same if previously described herein.

The figures provided herein are for illustrative purposes and may not be to scale. Variations in dimensions, proportions, and configurations may exist between the figures and the actual embodiments. The figures are intended to facilitate understanding of the embodiments and should not be construed as limiting the scope of the disclosure.

### DESCRIPTION OF EMBODIMENTS

This application describes exemplary transducer apparatuses capable of delivering TTFields to a subject's body for treating one or more cancers.

Transducers used to apply TTFields to a subject's body may include multiple electrode elements electrically coupled together on a substrate and attached to the subject's body at a desired location, for example, via an adhesive backing of the substrate or a separately applied adhesive. Conventional transducers have large, rectangular surfaces so as to maximize a number of electrode elements that are located on the transducer for applying TTFields to the subject's body. However, subjects can experience skin irritation on portions of their skin that are contacted by the electrode elements during TTFields treatment. Such irritation may be common at positions directly underneath the electrode elements, where heat and current may be at their highest concentrations, particularly for electrodes around the outer edge of the array.

As recognized by the inventors, on transducer arrays that comprise multiple electrode elements, the portions of the transducer arrays positioned directly beneath the electrode elements may become hotter than the portions of the transducer arrays positioned between the electrode elements. Furthermore, higher currents flow through the electrode elements that may be located along the edge of the array compared to the electrode elements located toward the middle of the array. Further still, an electrode element located at a corner or similar sharp bend in the edge of the array may have a higher current than other electrode elements along the edge and near the center of the array.

As recognized by the inventors, an uneven distribution of current through the transducer array may lead to higher temperature zones (or “hot spots”), e.g., at the corners or edges of the transducer array, which, in turn, may limit the maximum operational current that may be driven by a transducer array and, as a result, the strength of the resulting TTFields.

However, it has long been a need to be able to increase the current for TTFields treatment since it has been shown that increased current improves the efficacy of the treatment. But, increasing the current produces hot spots around the array and, in practice, the current has been limited in order to stay below the regulated temperature limits on the patient's skin. Introduction of an anisotropic material layer (such as a sheet of graphite) in the array has allowed the spread of heat/current over the area of the anisotropic material layer and this reduces hot-spots. Greater contiguous areas of anisotropic material layer are considered to be advantageous for spreading heat/current, thereby reducing hot spots (in turn allowing treatment with a higher current).

The inventors have now recognized that a need exists for transducer apparatuses with greater flexibility for comfortably contouring to a subject's body. In particular, the anisotropic material layer (for example, graphite sheet) is inflexible within the plane of the array of electrode subassemblies. In addition, the inventors have now recognized that a need exists for transducer apparatuses that can easily fit around certain anatomical features, chemotherapy ports, and/or other areas to be avoided on the subject's body. Transducer apparatuses that are able to effectively contour to a subject's body while avoiding areas that need to remain uncovered and/or would otherwise cause discomfort can be placed at an optimal location on the subject's body. As a result, the transducer apparatuses can induce TTFields through the subject's body at an ideal location and power level for targeting a region of interest (e.g., tumor) in the subject's body, thereby improving patient outcomes.

As discovered by the inventors, the transducer apparatuses, when viewed from a direction perpendicular to the transducer apparatuses, may also have an areal footprint and, in some embodiments, a U-shaped, V-shaped, rounded V-shaped, C-shaped, annular shaped, substantially U-shaped, substantially V-shaped, substantially rounded V-shaped, substantially C-shaped, or substantially annular shaped areal footprint. With such an areal footprint, the transducer apparatuses may be readily contoured to the anatomical shape of the subject's body without needing to be reconfigured or placed in a sub-optimal location. The areal footprints of the disclosed transducer apparatuses may provide greater flexibility and continuity for transducer placement around areas such as a breast, ear, chemotherapy port, surgical scar, skin lesion, or any other shaped area on the subject's body that may be difficult for the placement of transducers without causing discomfort.

Furthermore, the inventors have discovered that some transducer apparatuses may benefit from additional flexibility to better contour and adhere to the surface of a subject's body. To this end, the inventors have discovered that segmenting the anisotropic material layer of a transducer apparatus may allow greater flexibility than a non-segmented anisotropic material layer in that the transducer apparatus may flex at the divisions between the segments (sections) of anisotropic material layer. By contrast, a non-segmented anisotropic material layer may be too rigid to flex meaningfully, which may lead to the transducer with a non-segmented anisotropic material layer not completely adhering to the subject's body and/or pealing or pulling away from the subject's body during activity, movement, and/or shifting contours of the subject's body.

The inventors have discovered comfort and usability benefits associated with a sectioned transducer apparatus configuration. For example, the inventors have discovered that a non-segmented transducer apparatus may trap more heat, humidity, and/or sweat between the transducer apparatus and the subject's skin, thereby causing maceration and/or other forms of irritation and discomfort. As discovered by the inventors, by segmenting the transducer apparatus into two or more smaller segments, the heat, humidity, and/or sweat may be better ventilated, thereby possibly preventing maceration, irritation, and/or discomfort for the subject. The inventors have further discovered that segmentation of the transducer apparatus may not substantially impact heat distribution of the transducer apparatus and/or may not substantially contribute to the creation of hot spots on the subject's skin. Providing segments (sections) of anisotropic material layer on a single transducer array substrate takes advantage of the flexibility and breathability of the substrate (typically a medical tape or bandage) which is present between the segments of anisotropic material layer. However, alternative embodiments place each segment of anisotropic material layer on a separate portion of substrate, each shaped and contoured to match that of each segment of anisotropic material layer. In the latter embodiment, only the flexible electrical connector bridges the gap between adjacent substrate/segment of anisotropic material layer. This configuration offers the possibility of securing the transducer array in place on the subject's body without any conventional adhesive that could contribute to skin irritation. Instead, the front face of the segments of anisotropic material layer may use a non-adhesive conductive silicone elastomer to secure the transducer array in place and an elasticated band (e.g., a belly band) to hold the constructs in position in the event that extreme twisting/stretching movements momentarily release and reattach the non-adhesive conductive silicone elastomer front face of the array.

The inventors have further discovered that comfort and usability may be improved by providing segments of anisotropic material layer which are coincident with electrode subassembly units that are rotationally symmetric. In this way, when a subject experiences discomfort, the subject may remove the transducer apparatus and rotate and/or reposition the transducer apparatus as needed (e.g., by moving the electrodes into positions previously occupied by gaps between the electrodes, and vice-versa). Additionally, providing segments of anisotropic material layer as opposed to a continuous sheet that covers the array (or majority of the array) may aid in breathability of the array in the manner discussed above.

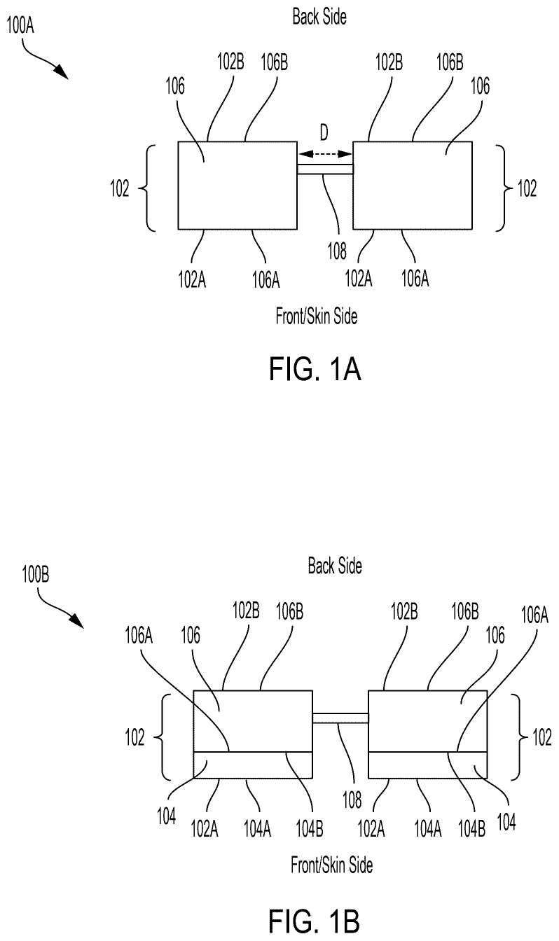

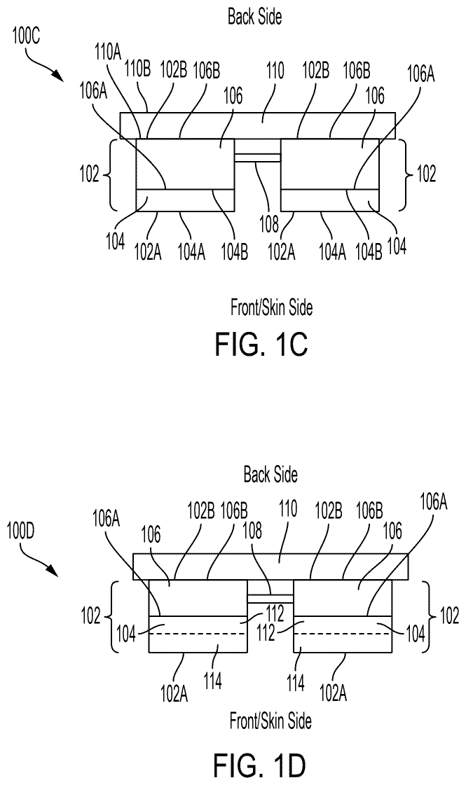

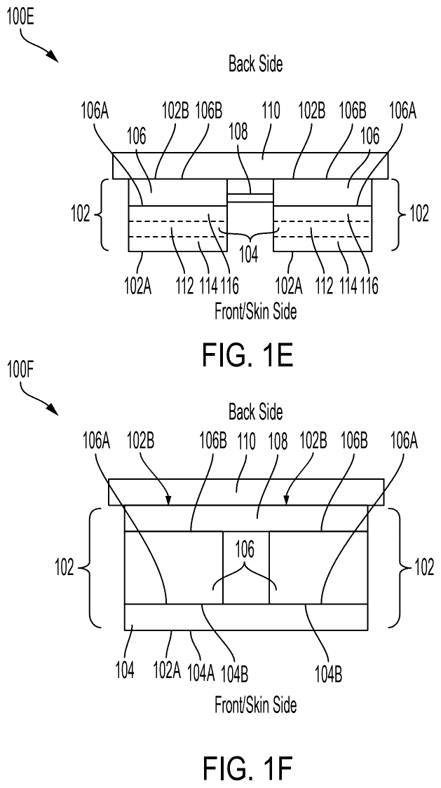

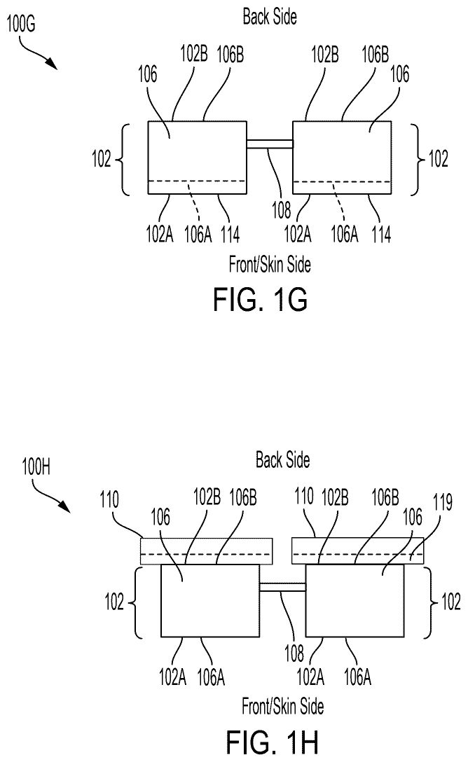

FIGS. 1A-1H depict cross-sectional views of exemplary transducer apparatuses. FIGS. 1A-1H each depict a cross-sectional view of an example transducer apparatus (generally referred to as transducer apparatus 100) which may include a number of components. For example, FIG. 1A depicts a transducer apparatus 100A which includes at least two electrode subassemblies 102 which are electrically connected by a flexible electrical connector 108. Although FIG. 1A depicts a transducer apparatus 100A which includes two electrode subassemblies 102, one having ordinary skill in the art will understand that any number of electrode subassemblies 102 may be used. For example, some embodiments may include 2, 3, 4, 5, 6, 7, 8, 9, 10, 11, 12, 13, 14, 15, 16, 17, 18, 19, or 20 or more electrode subassemblies 102, each electrode subassembly 102 comprising at least one electrode element 106. (Hereinafter, the electrode element 106 will be generally referred to in the singular for clarity; however, one having skill in the art will understand that electrode subassembly 102 may include any suitable number of electrode elements 106.) Flexible electrical connector 108 may be, for example and without limitation, a flexible printed circuit board (PCB) or a portion thereof, or wiring. As seen in FIG. 1A, the electrode subassemblies 102 may each include a front face 102A and a back face 102B (and the electrode elements 106 may each include a front face 106A and a back face 106B). The front face 102A may face the subject when the transducer apparatus 100 is affixed to the subject, and the back face 102B may face away from the subject when the transducer apparatus 100 is affixed to the subject. As can be seen, due to the positioning of the flexible electrical connector 108, the electrode subassemblies 102 may be spaced away from each other. The electrode subassemblies 102 may also be movable relative to each other. For example,, the electrode subassemblies 102 may be spaced apart by a distance D, which may be any suitable distance. For example, in some embodiments, distance D may be 1 mm, 2 mm, 3 mm, 4 mm, 5 mm, 6 mm, 7 mm, 8 mm, 9 mm, 10 mm, 20 mm, 50 mm, or any other distance therebetween or greater.

FIG. 1B depicts a transducer apparatus 100B which includes at least two electrode subassemblies 102 which are electrically connected by a flexible electrical connector 108. Transducer apparatus 100B is analogous to transducer apparatus 100A (and with a similar labelling scheme), although, additionally, electrode subassemblies 102 may each include an anisotropic material layer 104 and at least one electrode element 106 which is electrically coupled to the anisotropic material layer 104. Other details regarding the anisotropic material layer 104 and properties thereof are described elsewhere, such as, for example, in U.S. Patent Application Publication No. 2023/0037806, which is hereby incorporated herein by reference in its entirety.

Anisotropic material layer 104 may include a front face 104A and a back face 104B, and each electrode element 106 may include a front face 106A and a back face 106B. As can be seen in FIG. 1B, the anisotropic material layer 104 may be positioned on the front side of the electrode element 106. In this way, the back face 104B of the anisotropic material layer 104 may be positioned on the front side of the front face 106A, or adjacent to the front face 106A, of the electrode element 106. In addition, the front faces 104A, 106A of the anisotropic material layer 104 and electrode element 106, respectively, may each face the subject's body, and the back faces 104B, 106B of the anisotropic material layer 104 and electrode element 106, respectively, may each face away from the subject's body.

In some embodiments, the sections of anisotropic material layers 104 and the respective electrode subassemblies 102 may be separated such that they are spaced apart and do not touch. In other embodiments, a section of an anisotropic material layer 104 may extend to overlap more than one electrode subassembly 102. Further details of segmented anisotropic material layers 104 are provided below.

In some embodiments, the anisotropic material layer 104 and the electrode subassembly 102 may each have an areal footprint. In some embodiments, the respective areal footprint may be the shape of the anisotropic material layer 104, and the shape of the electrode subassembly 102, respectively, when viewed from a direction perpendicular to the front face 102A of the electrode subassembly. In some embodiments, the areal footprint of the anisotropic material layer 104 may partially or wholly overlap the areal footprint of the electrode subassembly 102. In some embodiments, the areal footprint of the anisotropic material layer 104 may extend laterally beyond the areal footprint of electrode subassembly 102.

In some embodiments, the areal footprint of the electrode subassembly 102 is approximately equal to the areal footprint of the electrode element 106 (for example, in some instances when the electrode subassembly 102 comprises one electrode element 106).

In some embodiments, the anisotropic material layer 104 and the electrode element 106 may each have an areal footprint. In some embodiments, the respective areal footprint may be the shape of the anisotropic material layer 104 and the shape of the electrode element 106 when viewed from a direction perpendicular to the front face 102A of the electrode subassembly. In some embodiments, the areal footprint of the anisotropic material layer 104 may partially or wholly overlap the areal footprint of the electrode element 106. In some embodiments, the areal footprint of the anisotropic material layer 104 may extend laterally beyond the areal footprint of electrode element 106.

As mentioned above, in some embodiments the electrode subassembly 102 may include two or more electrode elements 106. In such embodiments, the collective areal footprint of the electrode elements 106 may include the areal footprints of all of the individual electrode elements 106. In some embodiments, the areal footprint of the anisotropic material layer 104 may partially or wholly overlap the collective areal footprint of the electrode elements 106. In some embodiments, the areal footprint of the anisotropic material layer 104 may extend laterally beyond the collective areal footprint of electrode elements 106.

FIG. 1C depicts transducer apparatus 100C, which is similar to the transducer apparatus 100B of FIG. 1B, except that transducer apparatus 100C includes substrate 110. Substrate 110 may assist in holding together various components of the transducer apparatus 100C. In some embodiments, the transducer apparatus 100C may be affixed to the subject's body with the aid of the substrate 110. Suitable materials for the substrate 110 may include, for example, cloth, foam, flexible plastic, and/or a conductive medical gel or adhesive. In some embodiments, the substrate 110 may take the form of an adhesive bandage (e.g., a medical bandage) in which case the substrate 110 may extend beyond the perimeter of the other transducer components and may be involved in adhering the transducer apparatus to the subject's body. In other words, when viewed from a direction perpendicular to the front faces of the plurality of electrode subassemblies 102, each of the plurality of electrode subassemblies 102 is positioned within the outer perimeter of the substrate 110. In other embodiments, the substrate 110 may not extend beyond the perimeter of the other transducer components and may not be involved in adhering the transducer apparatus to the subject's body. Substrate 110 may further include a front face 110A and a back face 110B which is opposite the front face 100B. Substrate 110 may be positioned adjacent to the electrode subassemblies 102 such that the front face 110A of the substrate 110 is adjacent to the back face 102B of the electrode subassemblies 102.

FIGS. 1D and 1E depicts transducer apparatuses 100D and 100E, respectively, which are similar to the transducer apparatus 100C of FIG. 1C, except that transducer apparatuses 100D and 100E include further options regarding the electrode subassembly 102. The anisotropic material layer 104 may be any conductive layer having different thermal and/or electrical conductivities in a direction perpendicular to the front face 104A of the anisotropic material layer 104 than in directions that are parallel to the front face 104A. The anisotropic material layer 104 may be anisotropic with respect to electrical conductivity properties, anisotropic with respect to thermal conductivity properties, or both. This allows the anisotropic material layer 104 to spread out current and/or heat over a larger surface area. In each case, these properties may help to lower the temperature of hot spots and/or help to raise the temperature of cooler regions when a given AC voltage is applied to the electrode subassembly 102. Accordingly, the current may be increased without exceeding a safety temperature threshold at any point on the subject's skin. Delivering a higher current has been shown to improve the efficacy of the treatment.

In some embodiments, such as shown in FIG. 1D, an additional layer may be present on the skin-facing side of, and in electrical contact with, the anisotropic material layer 104. In some embodiments, the additional layer may be a biocompatible skin-contact conductive adhesive layer 114. For example, in some embodiments, the anisotropic material layer 104 may be a graphite layer 112. The graphite layer 112 may be a graphite sheet such as a sheet of pyrolytic graphite, graphitized polymer film, a graphite foil made from compressed high purity exfoliated mineral graphite, or some other material. The skin-contact conductive adhesive layer 114 may be a conductive gel or hydrogel (such as a conductive medical gel or hydrogel) or a conductive adhesive or a conductive silicone elastomer. The conductive adhesive may be, for example, an acrylic or silicone adhesive with conductive particles (metal particles, carbon particles, etc.) dispersed therein; and the conductive silicone elastomer may be a non-adhesive silicone elastomer having conductive particles dispersed therein. In some embodiments, one or more of the electrode subassemblies 101, 103 may comprise a non-adhesive conductive skin-contact layer (i.e. mechanical adhesive). For example, the electrode subassemblies may comprise a non-adhesive conductive silicone skin-contact layer (e.g., a conductive silicone elastomer) positioned on the front side of the anisotropic material layer 104, wherein the layer of conductive silicone has a non-adhesive front face that is textured in a manner that makes the front face of the layer of conductive silicone adhere to human skin. The non-adhesive skin-contact layer may be a mechanical adhesive layer having a suction-type quality to allow for a momentary release and re-adhesion of the subassemblies in the event that movement or twisting of the body creates stresses at the skin-subassembly interface. Such mechanical adhesives may utilize microstructures or nanostructures that function similarly to the setae and spatulae found on the foot pads of certain reptiles, which may allow for relatively strong adhesion to a wide range of surfaces. The microstructures may be arranged in patterns that optimize the surface area and contact with surfaces, thereby enhancing adhesion through van der Waals forces. The adhesive structures may be fabricated from materials such as polymers, (e.g., elastomers), or composites, which may provide flexibility and durability. Such non-adhesive conductive silicone skin-contact material (i.e., mechanical adhesives) may be available as ElectroSkin Gecko product (available from Nanoleq, 8153 Rümlang, CH). The skin-contact conductive adhesive layer 114 may be in direct contact with the skin-facing side of the anisotropic material layer 104, or, in some embodiments, there may be an intervening layer between the anisotropic material layer 104 and the skin contact conductive adhesive layer 114. For example, in some cases there may be an intervening layer of conductive fabric between the anisotropic material layer 104 and the non-adhesive conductive silicone skin contact layer. The skin contact conductive adhesive layer 114 and non-adhesive conductive silicone skin contact layer discussed here may be suitable for any of the embodiments disclosed herein.

In some embodiments, such as shown in FIG. 1E, an additional layer may be present on the outward-facing side of, and in electrical contact with, the anisotropic material layer 104. This additional layer may facilitate the electrical contact between the electrode element(s) 106 and the anisotropic material layer 104 and may also be a conductive adhesive layer 116. In some embodiments, conductive adhesive layer 116 may be a conductive adhesive or conductive gel. Compositionally, in some embodiments, the same types of conductive adhesive may be suitable for both additional layer 114 and additional layer 116, and may be, in some cases, the same conductive adhesive. In other embodiments, additional layer 114 and additional layer 116 may be different. In some embodiments, conductive adhesive layer 116 may be a double-sided tape, such as 3M™ 1552 Clear Tape. The conductive adhesive layer 116 may be in direct contact with the outward-facing side of the anisotropic material layer 104. In some embodiments, the skin-contact conductive adhesive layer 114 may be in direct contact with the skin-facing side of the anisotropic material layer 104 and the conductive adhesive layer 116 may be in direct contact with the outward-facing side of the anisotropic material layer 104 thereby forming a 3-layer construct. Furthermore, in some embodiments, conductive adhesive layer 116 may have a greater adhesive strength than an adhesive strength of skin-contact conductive adhesive layer 114. In this way, inadvertent delamination of the graphite layer 112 (for example, caused by twisting or stretching movement of the subject) may be prevented because the weaker skin adhesive layer 114 may release the transducer apparatus 100 from the subject's body before the stronger conductive adhesive layer 116 can cause delamination to occur.

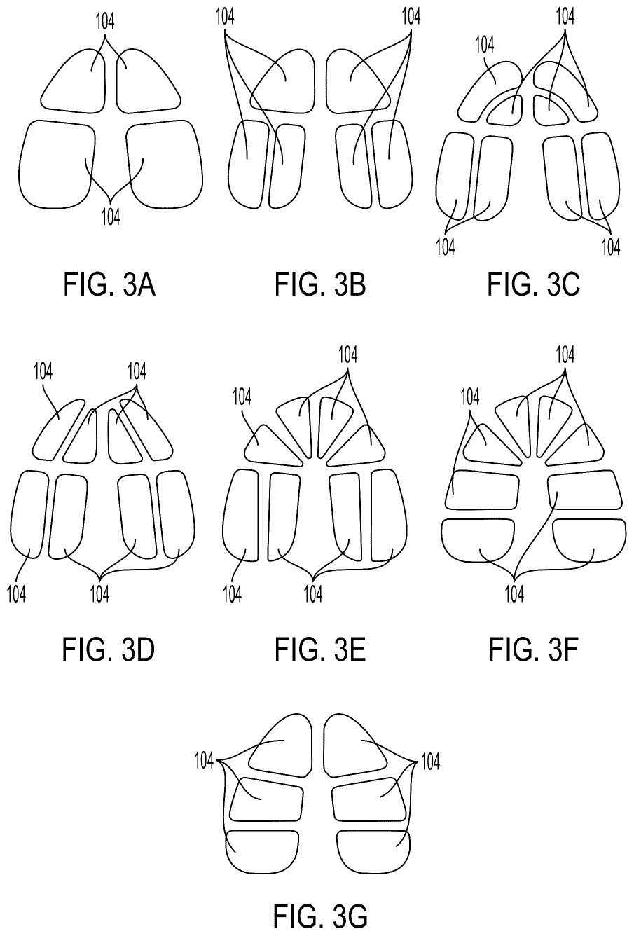

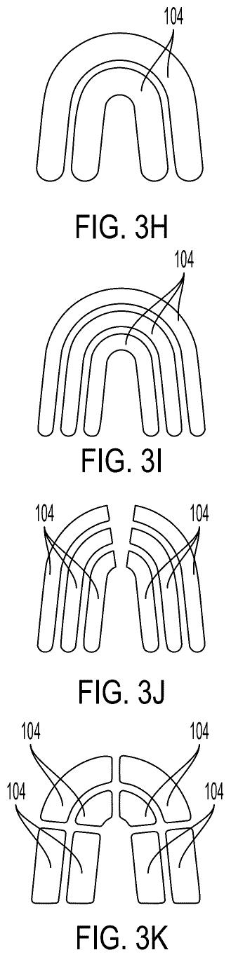

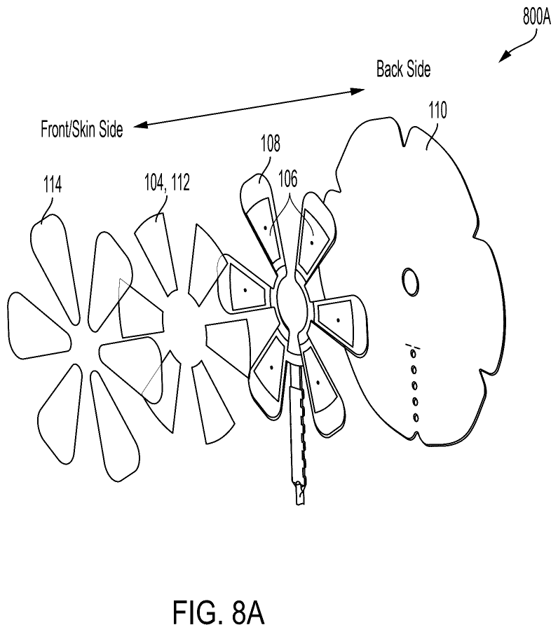

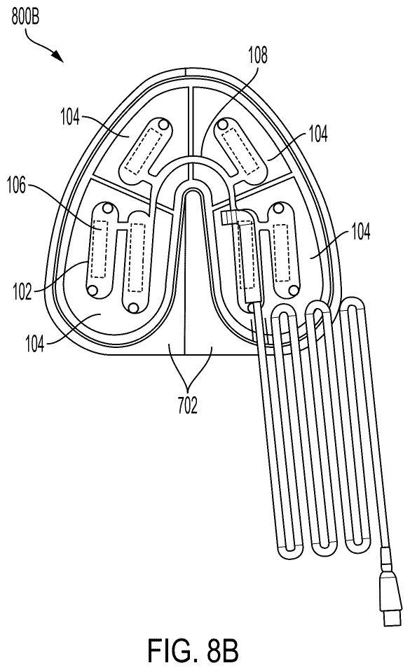

FIG. 1F depicts transducer apparatus 100F, which is similar to the transducer apparatuses 100B-100E of FIGS. 1B-1E, except that transducer apparatus 100F includes at least one section of anisotropic material layer 104 spanning across at least two of the plurality of electrode subassemblies 102. (Other electrode subassemblies 102 of the plurality of electrode subassemblies are not shown in the FIG. 1F). For example, the transducer array in FIG. 8B shows 6 electrode elements and 6 electrode subassemblies. FIG. 8B illustrates the use of 4 sections (segments) of anisotropic material layer 104, but, in fact, one could choose any of the configurations of sections (segments) of anisotropic material layer 104 shown in FIGS. 3A-3G. The configuration of sections (segments) of anisotropic material layer 104 shown in FIG. 3B provides one section of anisotropic material layer 104 for each of the 6 electrode subassemblies of FIG. 8B, whereby the areal footprint of each anisotropic material layer 104 wholly overlaps the areal footprint of the respective electrode subassembly 102 (in this case, the areal footprint of each anisotropic material layer 104 extends laterally beyond the areal footprint of each electrode subassembly 102). In contrast to the configuration of sections (segments) of anisotropic material layer 104 shown in FIG. 3B, FIG. 3A shows a configuration of 4 sections (segments) of anisotropic material layer 104 which could also be used for the transducer array of FIG. 8B. Each of the lower two sections (segments) of anisotropic material layer 104 in FIG. 3A would span across two of the electrode subassemblies 102 in FIG. 8B whereby the areal footprint of each anisotropic material layer 104 wholly overlaps the areal footprint of the combination of the two respective electrode subassemblies 102 (in this case also, the areal footprint of each anisotropic material layer 104 extends laterally beyond the areal footprint of the combination of the two electrode subassemblies 102). FIG. 1F is representative of the scenario described here (for the configuration of 4 sections (segments) of anisotropic material layer 104 shown in FIG. 3A which could be used for the transducer array of FIG. 8B, but illustrating only one of the lower two sections (segments) of anisotropic material layer 104).

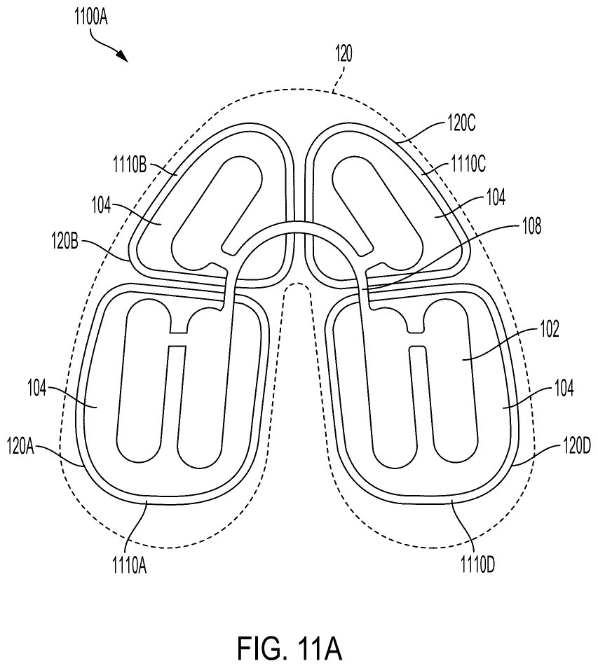

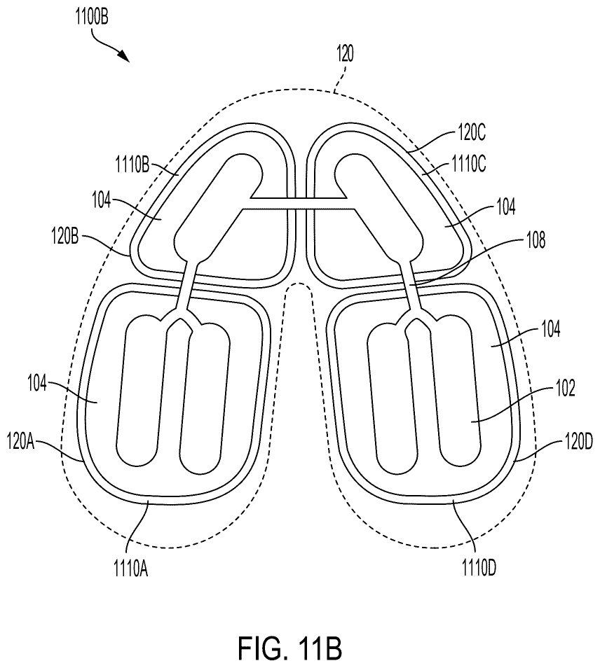

FIGS. 1G and 1H depict two transducer apparatuses 100G, 100H which, like transducer apparatus 100A, do not include anisotropic material layer 104. As can be seen in FIG. 1G, in some embodiments, adhesive layer 114 may be positioned on the front face 102A of the electrode subassemblies 102. As can be seen in FIG. 1H, in some embodiments, an adhesive layer 119 may be a part of substrate 110 such that adhesive layer 119 is positioned on the back face 102B of the electrode subassemblies 102. Adhesive layer 119 may aid in adhering the substrate to the subject's skin and thereby hold the electrode subassemblies 102 in place. An additional skin contact conductive adhesive 114 may also be present on the front face(s) of the electrode subassemblies 102 (not shown in 1H). Adhesive layer 119 need not be a conductive adhesive, although, preferably, adhesive layer 119 should be a biocompatible adhesive. As can also be seen in FIG. 1H, some embodiments may include separate substrates 110 for each respective electrode subassembly 102, which will be discussed in further detail in relation to FIGS. 11A and 11B. Although not depicted in FIG. 1H, some embodiments may include a single substrate 110 as is shown in, for example, FIG. 1C.

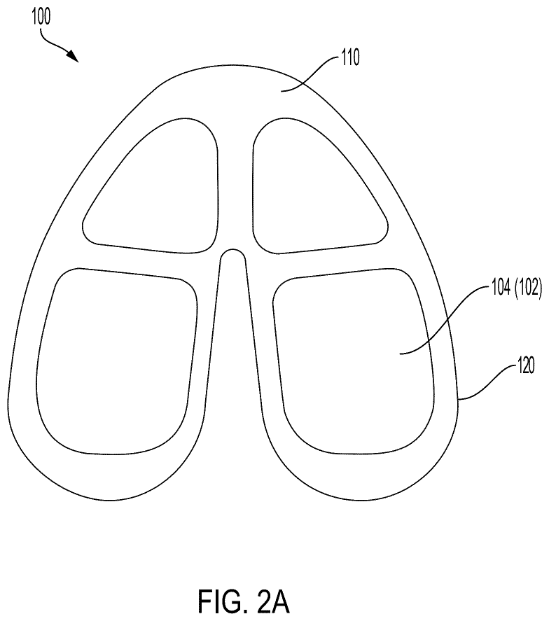

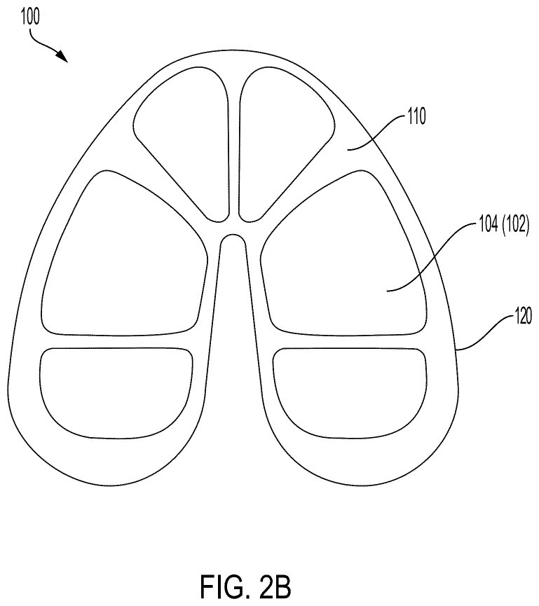

FIGS. 2A and 2B depict exemplary arrangements of separated, segmented anisotropic material layers for use in a transducer apparatus, when viewed from the direction perpendicular to (and toward) the front face 104A of the anisotropic material layer 104. As explained above, it may be desirable to provide a number of differently-shaped transducer apparatuses 100 to accommodate different placement positions on the body of the subject. FIGS. 2A and 2B each depict transducer apparatuses 100 having a rounded V-shape, but other shapes may also be possible, as will be explained in further detail below. FIGS. 2A and 2B also include substrate 110 which may, as explained above, extend beyond the perimeter of the other transducer components and may be involved in adhering the transducer apparatus to the subject's body. Furthermore, substrate 110 may include and/or define an areal footprint 120. In some embodiments, areal footprint 120 of substrate 110 may represent the projected two-dimensional area that substrate 110 occupies on a surface (e.g., on the subject's body), essentially representing the shape and size of the outer boundary (outer perimeter of the substrate) when viewed from above or perpendicular to the surface the substrate 110 rests upon.

As discussed above, the anisotropic material layer 104 may possess some out-of-plane flexibility (i.e., bending), but cannot stretch in-plane, nor does the sheet of anisotropic material layer 104 have any significant breathability (for air or moisture). Accordingly, a single sheet of anisotropic material layer 104 restricts the flexibility of the transducer array and also fails to allow moisture (e.g., from sweating) from dissipating from the skin under the transducer apparatus. Presenting the anisotropic material layer 104 as segments that make up the desired transducer array shape may improve the flexibility and breathability of the transducer array. Because the substrate is generally a medical tape or bandage selected for its flexibility and breathability, the inter-segment areas (substrate) may provide that flexibility and breathability. The shapes and relative orientation of the anisotropic material layer 104 segments can be selected to optimize such properties. For example, in FIG. 2A, the inter-segment gap between the anisotropic material layer 104 segments near the apex (in the area of the join of the rounded-V) allows the two arms of the rounded-V to be stretched apart slightly, thereby relieving stresses in the transducer array (and the subject's skin) when the subject twists and the skin stretches.

As described above, a skin-contact conductive adhesive may be present on the skin-facing side of the anisotropic material layer 104 segments, and/or the transducer array may be adhered to the subject's skin via an adhesive on the skin-facing side of the substrate. Also described herein, a foam perimeter framing the transducer array may have an adhesive on the skin-facing side. All of these embodiments may be applicable and find use in the FIGS. 2A and 2B embodiments, or indeed any of the other embodiments described herein. Furthermore, embodiments such as those of FIGS. 2A and 2B may function without the use of any conventional adhesive. For example, the FIGS. 2A and 2B embodiments may utilize a non-adhesive conductive silicone elastomer to help affix the transducer array to the subject, and a non-adhesive elasticated wrap-around bandage (e.g., a belly band) can hold the transducer array in place. The transducer array can absorb excessive twisting of the subject's body by momentarily detaching and reattaching to the skin via the non-adhesive conductive silicone elastomer while the elasticated wrap-around bandage holds the transducer array generally in place. This type of embodiment is envisioned for all of the embodiments disclosed herein.

As explained above in connection with FIGS. 1A and 1G-1H, some embodiments may not include anisotropic material layer 104. In such embodiments, the segments depicted FIGS. 2A and 2B may instead represent electrode subassembly 102 which would otherwise be obscured from view by anisotropic material layer 104. One having ordinary skill in the art will also understand that such an embodiment applies equally to all embodiments discussed and depicted herein.

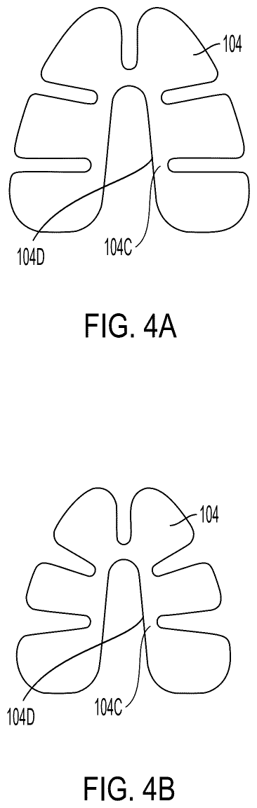

FIGS. 3A-3K depict exemplary arrangements of segmented anisotropic material layers for use in a transducer apparatus. For visual clarity, substrate 110 has been omitted from FIGS. 3A-3K (and FIGS. 4A-6H), but one having ordinary skill in the art will understand that all embodiments discussed and depicted herein may or may not include substrate 110. As explained above, it may be desirable to provide a number of differently-shaped transducer apparatuses 100 to accommodate different placement positions on the body of the subject. For one such transducer shape (for example, a substantially rounded V-shape transducer apparatus, such as the one shown in FIG. 8B), FIGS. 3A-3K each depict a different layout for the collective areal footprints of anisotropic material layer 104 segments. As shown in FIGS. 3A-3K, each of the collective areal footprints of anisotropic material layer 104 segments defines a shape which may be described as substantially U-shaped, substantially V-shaped, and/or substantially rounded V-shaped. Substantially C-shaped transducer arrays may have similar arrangements of anisotropic material layer 104 segments. As discussed above, the anisotropic material layers 104 may be segmented such that they are spaced apart and do not touch.

FIGS. 4A-4B depict exemplary arrangements of segmented anisotropic material layers for use in a transducer apparatus, wherein the segments are connected by thin bridges. In some embodiments, such as shown in FIG. 1F, the electrode subassemblies 102 may be spaced apart and do not touch, and the anisotropic material layer 104 are segmented but joined by a thin bridging portion. As shown in FIGS. 4A and 4B, the anisotropic material layer 104 may include a thin bridging portion between portions covering areal footprint of the electrode subassemblies 102. In some embodiments, the segmented anisotropic material layers 104 may be: sections of anisotropic material layers spaced apart and not touching; sections of anisotropic material layers connected to at least one other section of anisotropic material layer with a thin bridging anisotropic material layer; or when viewed from a direction perpendicular to the front face of the electrode subassemblies, the anisotropic material layers of the electrode subassemblies may define a contiguous shape with peninsula portions having an areal footprint sufficient to be coincident with one or more electrode subassembly. Further details of segmented anisotropic material layers 104 are provided below. FIGS. 4A-4B depicts different layouts for the areal footprints of anisotropic material layers 104. The embodiments of FIGS. 4A-4B differ from those of FIGS. 2A-2K in that the each “segment” of the anisotropic material layer 104 is connected to the adjacent “segment” by a thin bridge 104C, which in some embodiments may be defined by an interior edge 104D of the anisotropic material layer 104. In some embodiments, each thin bridging anisotropic material layer has a width less than one third the width of a smallest width of its two adjoining sections of anisotropic material layer. In this way, each segment of the anisotropic material layer 104 may define a peninsula-like portion. By shaping the anisotropic material layer 104 in this manner, some flexibility of the segmented design may be retained while also simplifying the manufacturing process.

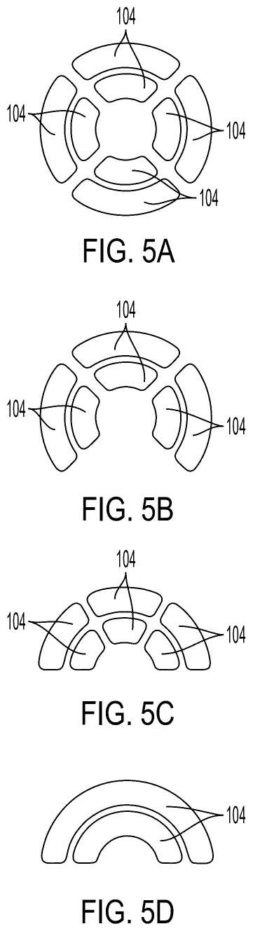

FIGS. 5A-5D depict exemplary arrangements of segmented anisotropic material layers for use in a transducer apparatus. FIGS. 5A-5D are similar to FIGS. 3A-3K in that each figure depicts a different layout for the collective areal footprints of anisotropic material layer 104 segments. Notably, the anisotropic material layer 104 segments of FIG. 5A define a substantially annular or circular shape. The anisotropic material layer 104 segments of FIG. 5B define a substantially C-shape. And the anisotropic material layer 104 segments of FIGS. 5C-5D define a substantially semi-annular shape or semi-circular shape.

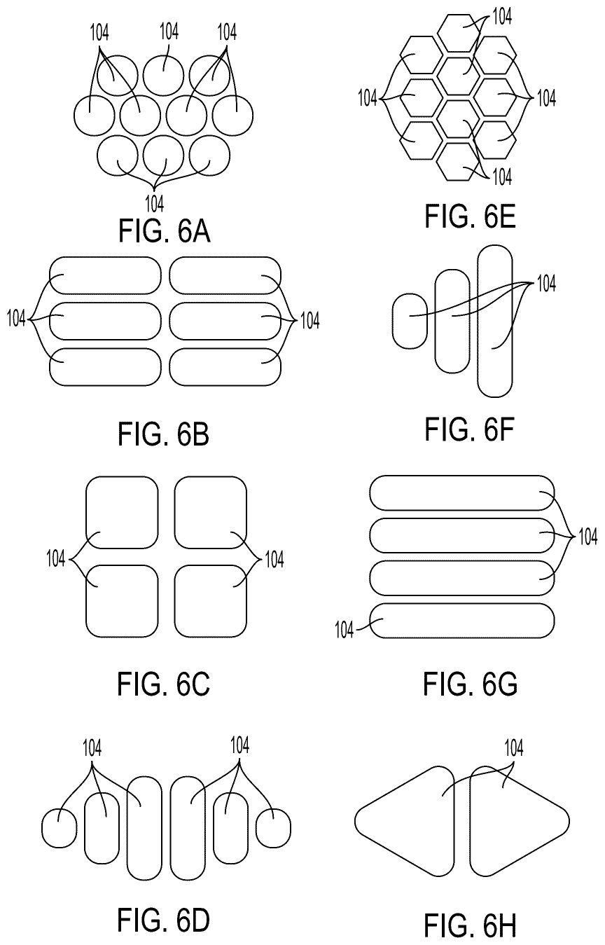

FIGS. 6A-6H depict exemplary arrangements of segmented anisotropic material layers for use in a transducer apparatus. FIGS. 6A-6H are similar to FIGS. 3A-3K and 5A-5D in that each figure depicts a different layout for the collective areal footprints of anisotropic material layer 104 segments. FIGS. 6A-6H differ from FIGS. 3A-3K and 5A-5D in that the collective areal footprints define various polygons. For example, FIGS. 6A and 6E each define a hexagonal or an octagonal shape. FIGS. 6B-6C and 6G-6H each define a quadrilateral shape. FIG. 6D defines a quadrilateral, a hexagonal, or an octagonal shape. FIG. 6F defines a triangular or quadrilateral shape. Although not pictured, in some embodiments the areal footprint can define a heptagonal shape. As will be understood by one having skill in the art, any other suitable polygonal shape may also be possible.

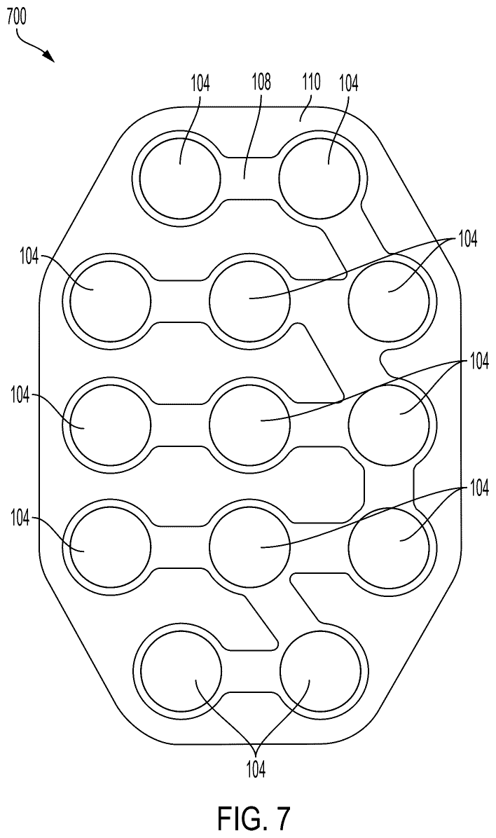

FIG. 7 depicts a transducer apparatus having an exemplary arrangement of anisotropic material layer segments viewed from the skin-facing side. For transducer apparatus 700 of FIG. 7, both the collective areal footprints of anisotropic materials 104 and substrate 110 define an octagonal shape. In FIG. 7, each of the 13 sections (segments) of anisotropic material layer 104 is coincident with an electrode subassembly 102 (the latter is not visible since each is wholly covered by a segment of anisotropic material layer 104).

FIGS. 8A-8B depict exemplary transducer apparatuses 800A, 800B having exemplary arrangements of anisotropic material layer segments 104 with flexible electrical connectors 108 connected thereto. In particular, FIG. 8A depicts an exploded view of transducer apparatus 800A. Transducer apparatus 800A may include anisotropic material layer 104 (graphite layer 112), skin-contact conductive adhesive layer 114, and electrode elements 106 which are disposed on flexible electrical connector 108 which, in this embodiment, may be a flexible PCB. Transducer apparatus 800A may also include substrate 110. Notably, transducer apparatus 800A differs from other transducers described to this point in that the areal footprint of the segments of anisotropic material layer 104 defines a star shape such that each of the anisotropic material layer 104 segments are rotationally symmetric about a centroid defined thereby (more details on this aspect are provided below). Also notably, each segment of anisotropic material layer 104 corresponds to a respective segment of flexible electrical connector 108. In other words, flexible electrical connector 108 may be shaped to abut and/or align with the electrode subassemblies 102 and/or the electrode elements 106, and the various segments of anisotropic material layer 104 are positioned to wholly cover the electrode subassemblies 102 and/or the electrode elements 106. In some embodiments, the flexible electrical connector 108 may extend laterally beyond the areal footprint of the electrode element(s) 106 in some places. This may allow the segments of anisotropic material layer 104 to extend beyond the areal footprint of the electrode elements 106 and thereby allow spreading of the heat/current over a greater area. As such, in some embodiments, at least a portion of the flexible electrical connector 108 may be disposed adjacent to the back face of the respective anisotropic material layer 104.

FIG. 8B depicts a rear/outward side view of transducer apparatus 800B (with substrate 110 removed). The outward side is shown so that the segments of anisotropic material layer 104 do not obscure the electrode subassemblies 102. Transducer apparatus 800B is similar to transducer apparatus 800A, except that transducer apparatus 800B has a curved V-shape, which has been described above. Like transducer apparatus 800A in FIG. 8A, transducer apparatus 800B includes a flexible electrical connector 108 which is shaped to abut and/or align with the electrode subassemblies 102 and/or the electrode elements 106, and the various segments of anisotropic material layer 104 are positioned to wholly cover the electrode subassemblies 102 and/or the electrode elements 106. In FIG. 8B, the electrode elements 106 are shown in dash to represent them being between the flexible electrical connector 108 and the anisotropic material layer 104 and, as such, not being visible from this rear/outward side view of transducer apparatus 800B. Transducer apparatus 800B additionally includes peel-off cover 702 (release liner). All embodiments described and depicted herein may also include peel-off cover 702; however, peel-off cover 702 (release liner) has generally been omitted from the figures for visual clarity.

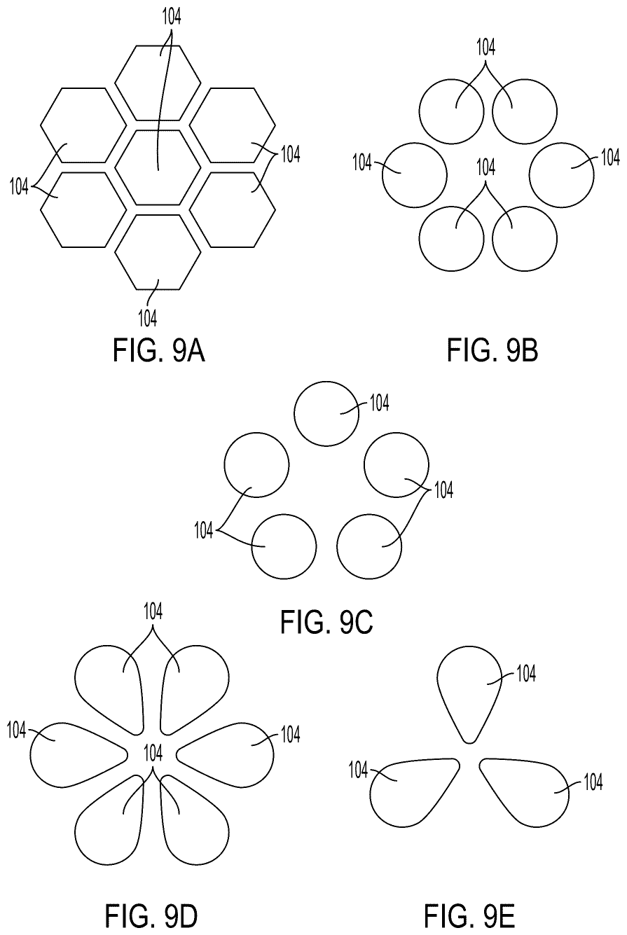

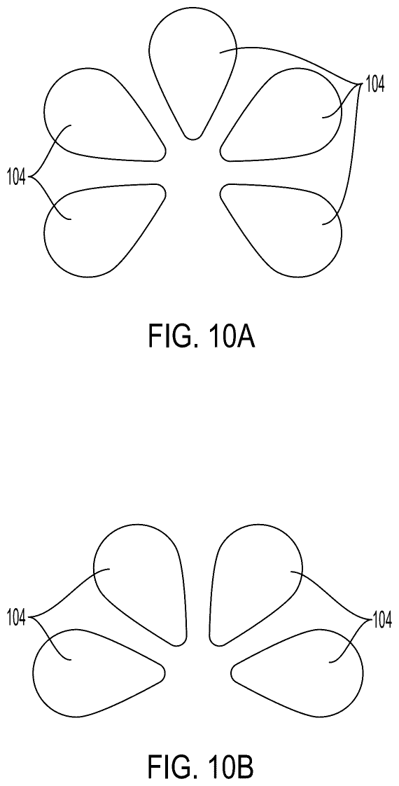

FIGS. 9A-9E depict exemplary arrangements of segmented anisotropic material layers for use in a transducer apparatus, where the segmented anisotropic material layers have the same shape and are rotationally symmetric. FIGS. 9A-10B are similar to FIGS. 3A-6H in that they depict a front view of anisotropic material layers 104 for various transducer configurations. As can be seen in each of FIGS. 9A-9E, each of the anisotropic material layers 104 may have the same shape, such as for example, a hexagon, a circle, a teardrop, or a petal shape. Also like the transducer apparatuses depicted in FIGS. 3A-6H, the anisotropic material layers 104 may be non-contiguous with respect to each other. Because of the orientation of the individual anisotropic material layers 104, it can be seen that the collective anisotropic material layers 104 of each transducer configuration may have a centroid, and may be rotationally symmetric about that centroid. In this way, should the subject experience discomfort from the transducer apparatus during use, the transducer apparatus may be easily removed, rotated, and/or otherwise adjusted, and reapplied. As has been described above, some transducer embodiments may benefit from having certain shapes (e.g., U-shaped, V-shaped, or rounded V-shaped) to better conform to the subject's body. In scenarios where this is not needed or not possible, however, the rotationally-symmetric style of FIGS. 9A-9E may allow for desired comfort for the subject.

FIGS. 10A-10B depict exemplary arrangements of segmented anisotropic material layers for use in a transducer apparatus, where the segmented anisotropic material layers have the same shape. FIGS. 10A-10B are similar to FIG. 9D, except that at least one anisotropic material layer 104 segment has been removed. For example, in in FIG. 9A, one anisotropic material layer 104 segment has been removed, and in FIG. 9B, two anisotropic material layer 104 segments have been removed. In this way, if a subject were to experience discomfort such as skin irritation or maceration, the discomfort may be alleviated by rotating the transducer such that the irritated portion of the skin is left uncovered by the one or more removed anisotropic material layer 104 segments.

As explained above in connection with FIG. 1H, in some embodiments, the substrate 110 may be segmented into separate substrate sections for each respective electrode subassembly 102. Such embodiments can be seen, for example, in FIGS. 11A and 11B. FIGS. 11A-11B depict substantially U-shaped or substantially rounded V-shaped transducer apparatuses 1100A, 1100B. Transducer apparatuses 1100A, 1100B are generally similar to each other but include different exemplary electrode subassembly 102 and PCB layouts. Transducer apparatuses 1100A, 1100B include separate substrate sections 1110A-1110D (collectively substrate 1110), and each electrode subassembly 102 of the transducer apparatuses 1100A, 1100B is associated with a distinct substrate section 1110A-1110D. In some embodiments, the substrate sections 1110A-1110D of the substrate 1110 may be separated, apart, not touching, not overlapping, and/or the like. In this way, the individual electrode subassemblies 102 may not be connected to each other by the substrate 1110—and may only be connected by flexible PCB 108—such that they are capable of moving and being repositioned independent of each other. As can also be seen, each substrate section 1110A-1110D includes an outer perimeter 120A-120D which is shaped and sized such that each electrode subassembly 102 may be positioned within the outer perimeter of its respective substrate section 1110A-1110D. In some embodiments, when viewed from a direction perpendicular to the front face of the electrode subassemblies, the substrate 1110 may be segmented into a plurality of sections, and the plurality of sections of the substrate may define a substantially C-shape, a substantially U-shape, a substantially V-shape, a substantially rounded V-shape, or a substantially annular shape. As can be seen in FIGS. 11A-11B, the areal footprint of the substrate 1110 is a substantially U-shaped, or substantially rounded V-shaped. For comparison, FIGS. 11A-11B depict in dashed lines the areal footprint 120 of substrate 110 from FIGS. 2A-2B, respectively, which is also a substantially U-shaped, or substantially rounded V-shaped. As can be seen, the areal footprint 120 is larger than the collective areal footprint of the substrate 1110.

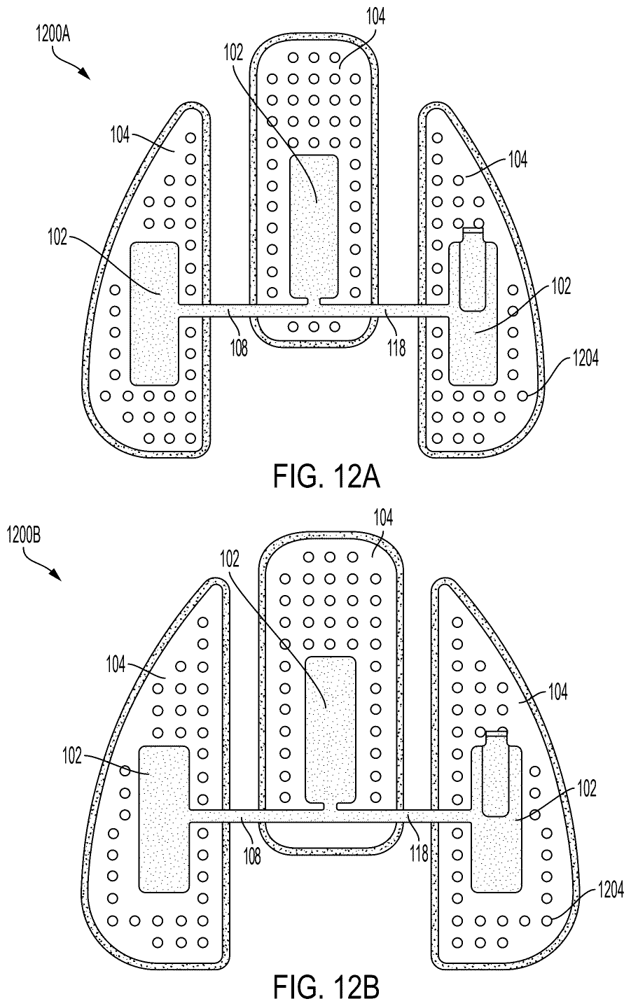

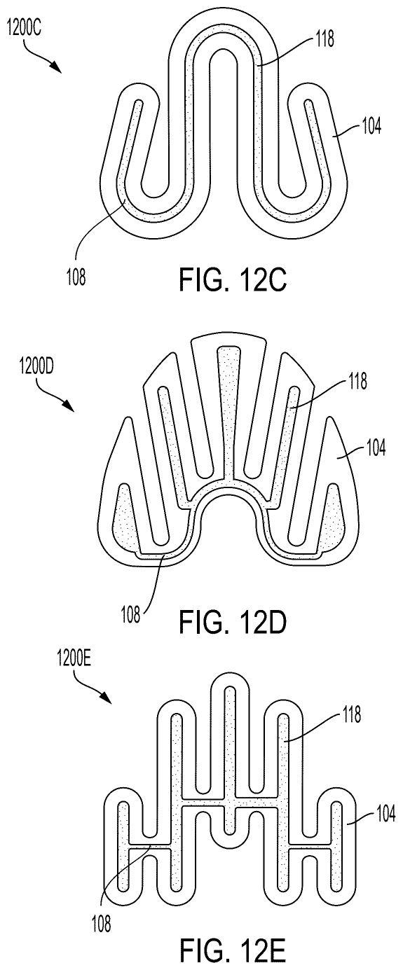

FIGS. 12A-12E depict additional, exemplary transducer arrays 1200A, 1200B, 1200C, 1200D, 1200E, respectively. Transducer apparatuses 1200A, 1200B are largely similar except that, when viewed from a direction perpendicular to the transducer apparatus, the anisotropic material layer 104 of transducer apparatus 1200B has an approximately 10% larger surface area than the anisotropic material 104 of transducer apparatus 1200A. As will be understood by one having ordinary skill in the art, a selection of the size of the surface area of the anisotropic material layer 104 may be considered a balance between the subject's comfort and the effectiveness of the TTFields.

Transducer apparatuses 1200A-1200E differ from others described and depicted herein in that the electrode subassemblies 102 are integrated within flexible electrical connector 108 such that they collectively form an electrode superassembly 118. As will be understood by one having ordinary skill in the art, flexible electrical connector 108 may be constructed with a polymer-based substrate that provides bendability and flexibility. This substrate may include materials such as polyimide or polyethylene terephthalate (PET), which allow for the required flexibility and thermal stability. Embedded within this flexible substrate, a plurality of electrode subassemblies 102 may be positioned. The electrode subassemblies 102 may include electrode elements 106 which may in turn be formed using conductive materials such as copper or silver, which may be deposited via techniques like etching, plating, or printing. The electrode subassemblies 102 and/or electrode elements 106 may be arranged in a predefined pattern to facilitate electrical connectivity and signal transmission across the flexible electrical connector 108. One having ordinary skill in the art will understand that, although certain embodiments disclosed herein refer to an electrode subassembly 102 which may include one or more electrode elements 106, other embodiments may include electrode superassembly 118, without necessitating any other structural or functional changes to said embodiments.

As can be seen in FIGS. 12C-12E, the transducer apparatuses 1200C, 1200D, 1200E and electrode superassembly 118 therein may have any number of shapes. As can be seen in FIGS. 12C-12E, in some embodiments, the transducer apparatuses 1200C, 1200D, 1200E and electrode superassembly 118 may have abstract shapes. In other embodiments, the transducer apparatuses 1200C, 1200D, 1200E and electrode superassembly 118 may have a substantially M-shape, a substantially N-shape, a substantially W-shape, a substantially O-shape, a substantially U-shape, a substantially V-shape, a substantially C-shape, or a substantially D-shape. One having ordinary skill in the art will understand that the particular size and shape of the transducer apparatuses 1200C, 1200D, 1200E and electrode superassembly 118 may be dictated by subject comfort, electrode efficacy, cost, and/or manufacturing concerns.

Transducer apparatuses 1200A, 1200B may also include perforation holes 1204 within anisotropic material layer 104. Perforation holes 1204 may aid in ventilating heat and/or moisture, and may be equally applied to any other embodiment disclosed herein.

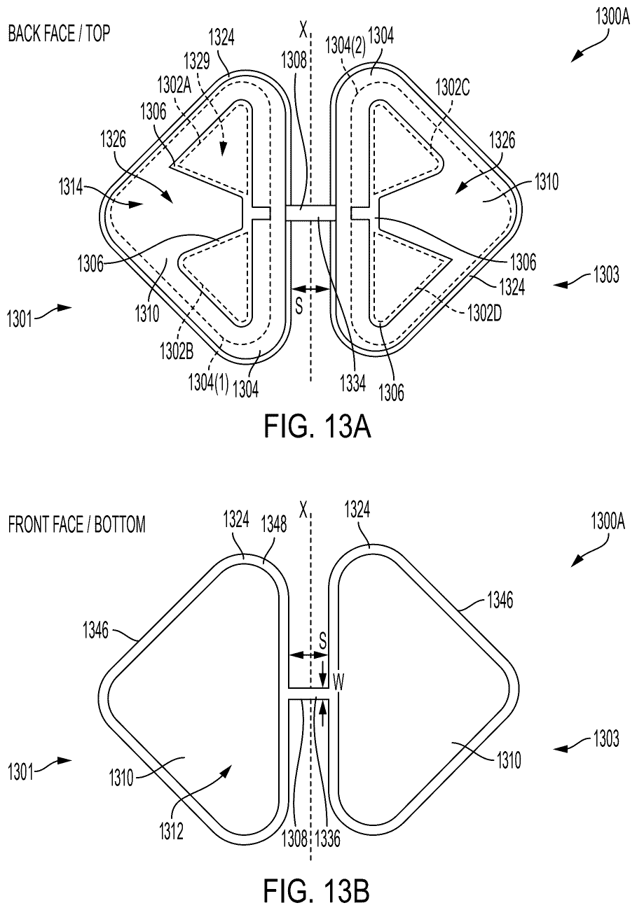

FIGS. 13A and 13B depict a top plan view (i.e., a back face plan view) and a bottom view (i.e., a front face view, or a skin-facing view), respectively, of an example transducer apparatus 1300A according to some embodiments. FIGS. 13A and 13B illustrate the transducer apparatus 1300A as viewed from a direction perpendicular to a face of the transducer apparatus 1300A. FIG. 13A illustrates a plan view from the back side of the transducer apparatus 1300A (i.e., the side facing away from the subject's body, or the back face, or the top). FIG. 13B illustrates the front side of the transducer apparatus 1300A (i.e., the side facing the subject's body, or the front face, or the bottom).

As shown in FIGS. 13A and 13B, the transducer apparatus 1300A may include a first electrode subassembly (or first transducer subassembly) 1301 and a second electrode subassembly (or second transducer subassembly) 1303. The first electrode subassembly 1301 and the second electrode subassembly 1303 may have shapes that are symmetrical along the sagittal plane X and include the same components. Thus the following description of the components apply to both the first and the second electrode subassemblies 1301, 1303.

Each of the first and the second electrode subassemblies 1301, 1303 may include at least one electrode element 1302 (i.e., 1302A, 1302B, 1302C, 1302D), a flexible printed circuit board (PCB) 1306 electrically connecting the electrode elements 1302, an anisotropic material layer 1310 electrically coupled to the at least one electrode element 1302, and a substrate 1304 for holding the at least one electrode element and the anisotropic material layer against the subject's body. The first and the second electrode subassemblies 1301, 1303 may be connected with a flexible electrical connector 1308, where the flexible electrical connector 1308 separates the first and the second electrode subassemblies 1301, 1303. In FIG. 13A, the electrode elements 1302A, 1302B, 1302C, and 1302D are shown in dashed outline, as they are located between the flexible PCB 1306 and the anisotropic material layer 1310. In FIG. 13A, although the substrate 1304 covers the back sides of the first and the second electrode subassemblies 1301, 1303, the substrate 1304 is shown as a cut-away in a partial view by the dashed lines 1304(1) and 1304(2) so as to illustrate the components beneath the substrate 1304.

When viewed in a direction perpendicular to and toward the back face of the subassemblies 1301, 1303 (as in FIG. 13A), the first and second electrode subassemblies 1301, 1303 may have a same shape (or mirror image thereof). In some embodiments, each of the first and second electrode subassemblies 1301, 1303 may be triangular shaped, substantially triangular shaped, rounded triangular shaped, substantially rounded triangular shaped, kidney bean or jelly bean shaped, substantially kidney bean or jelly bean shaped, ovoid shaped, substantially ovoid shaped, oval shaped, or substantially oval shaped, or each subassembly has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature. For example, in FIG. 13A, the first and second electrode subassemblies 1301, 1303 may be triangular or rounded triangular shaped.

In some embodiments, the first and the second electrode subassemblies 1301, 1303 may be the same size, have the same shape, have the same dimensions, and have the same surface area. In some embodiments, the first and the second electrode subassemblies 1301, 1303 may be different sizes and/or shapes. For example, one of the electrode subassemblies may have a surface area at least 25% and at most 50% larger (including any percentage therebetween) than the surface area of the other electrode subassembly.

Each of the first and the second electrode subassemblies 1301, 1303 may include at least one electrode element 1302. In some embodiments, each of the first and the second electrode subassemblies 1301, 1303 has at least two electrode elements 1302. In some embodiments, each of the first and the second electrode subassemblies 1301, 1303 has only one electrode element 1302. In some embodiments, the subassemblies 1301, 1303 have the same number of electrode elements 1302. In some embodiments, the subassemblies 1301, 1303 have a different number of electrode elements 1302.

The electrodes elements 1302 of the first and the second electrode subassemblies 1301, 1303 may be adapted to administer TTFields therapy to a subject. The electrodes elements 1302 may be electrically conductive and may be substantially flat. The electrode elements 1302 may or may not be capacitively coupled. The electrodes elements 1302 may be metal, metal alloy, layered metal, laminated conductive material, or may be ceramic or non-ceramic dielectric material positioned over a flat conductor (e.g., polymer film disposed on flat metal). In some embodiments, the electrode elements do not have a dielectric material.

The first and the second electrode subassemblies 1301, 1303 may include the same number or different numbers of electrodes elements 1302. In some embodiments, the first and second electrode subassemblies 1301, 1303 may include one, two, three, four, five, six, seven, eight, nine, ten, eleven, twelve, thirteen, fourteen, fifteen, sixteen, seventeen, eighteen, nineteen, twenty, or more electrodes elements 1302. For example, in FIG. 13A, the first and the second electrode subassemblies 1301, 1303 are depicted as having two electrodes elements each. The first electrode subassembly 1301 includes two electrodes 1302A, 1302B, and the second electrode subassembly 1303 includes two electrodes 1302C, 1302D.

In some embodiments, the electrodes elements 1302 may have a same shape. In some embodiments, when viewed in a direction perpendicular to and toward the back face of the subassemblies, the electrodes element 1302 may be triangular shaped, substantially triangular shaped, rounded triangular shaped, substantially rounded triangular shaped, kidney bean or jelly bean shaped, substantially kidney bean or jelly bean shaped, ovoid shaped, substantially ovoid shaped, oval shaped, or substantially oval shaped, or each subassembly has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature. For example, in FIG. 13A, the first and the second electrode subassemblies 1301, 1303 are depicted as having two triangular shaped (or rounded triangular shaped) electrodes elements each. The first electrode subassembly 1301 includes two triangular or rounded triangular shaped electrodes 1302A, 1302B, and the second electrode subassembly 1303 includes two triangular or rounded triangular shaped electrodes 1302C, 1302D.

In some embodiments, the dielectric material of the electrode elements 1302 may have a dielectric constant ranging from 10 to 50,000. In some embodiments, the layer of dielectric material comprises a high dielectric polymer material such as poly(vinylidene fluoride-trifluoroethylene-chlorotrifluoroethylene) and/or poly(vinylidene fluoride-trifluoroethylene-1-chlorofluoroethylene). Those two polymers are abbreviated herein as “Poly(VDF-TrFE-CTFE)” and “Poly(VDF-TrFE-CFE),” respectively. The dielectric constant of these materials is on the order of 40. In some embodiments, the polymer layer may be poly(vinylidene fluoride-trifluoroethylene-chlorotrifluoroethylene-chlorofluoroethylene) or “Poly(VDF-TrFE-CTFE-CFE).”

In some embodiments, the layer of dielectric material of the electrode elements 1302 comprises a terpolymer comprising polymerized units of monomers such as VDF, TrFE, CFE and/or CTFE in any suitable molar ratio. Suitable terpolymers include those, for example, having 30 to 80 mol % VDF, 5 to 60 mol % TrFE, with CFE and/or CTFE constituting the balance of the mol % of the terpolymer.

The flexible PCB 1306 may electrically couple together the electrode elements 1302. In some embodiments, the flexible PCB 1306 may cover the back faces 1329 of the electrode elements 1302. An edge of the electrode element 1302 may be offset inwardly from an edge of the flexible PCB 1306. In some embodiments, when viewed in a direction perpendicular to and toward the back face of the electrode subassemblies 1301, 1303, the flexible PCB 1306 may cover up to and including 15%, 20%, 30%, 40%, 60%, 80%, or any percentage therebetween of a back face 1314 of the anisotropic material layer 1310. The flexible PCB 1306 does not function as an electrode. The flexible PCB 1306 may be a non-adhesive region. In some embodiments, the flexible PCB 1306 may comprise a polyimide film, such as, for example, Kapton® (from DuPont, Wilmington, DE, USA) and conductive traces (for example, and without limitation, copper traces or traces of conductive ink, etc.). In some embodiments, the flexible PCB 1306 may include a fluropolymer wrapper.

The flexible electrical connector 1308 may extend between and electrically couple the first and the second electrode subassemblies 1301, 1303. The flexible electrical connector 1308 may electrically connect the at least one electrode element 1302 of the first electrode subassembly 1301 and the at least one electrode element 1302 of the second electrode subassembly 1303.

The flexible electrical connector 1308 may include a front face facing the subject's body and a back face opposite the front face. The front face of the flexible electrical connector 1308 may be free of adhesive material. When the subassemblies 1301, 1303 are viewed in cross-section, the flexible electrical connector 1308 may be adapted not to be adhesively held to the subject's body.

The flexible electrical connector 1308 may further include a foam material layer 1334 on the back face of the flexible electrical connector 1308 and/or a foam material layer 1336 on the front face of the flexible electrical connector 1308. In some embodiments, the flexible electrical connector 1308 may be or may include a plastic coated wire to electrically connect the first and the second electrode subassemblies 1301, 1303.

In some embodiments, when viewed in a direction perpendicular to and toward the back face of the subassemblies 1301, 1303 (as in FIG. 13A), the flexible electrical connector 1308 may have a substantially linear shape. In some embodiments, when viewed in a direction perpendicular to and toward the back face of the subassemblies 1301, 1303, the flexible electrical connector 1308 may have different shape, such as, for example: a U-shape, substantially U-shape, or rounded V-shape; a shape with two or more bends, such as an undulating shape, serpentine shape, wavelike shape, zig-zag shape; a shape with a combination of concave and convex portions; or a spiral shape.

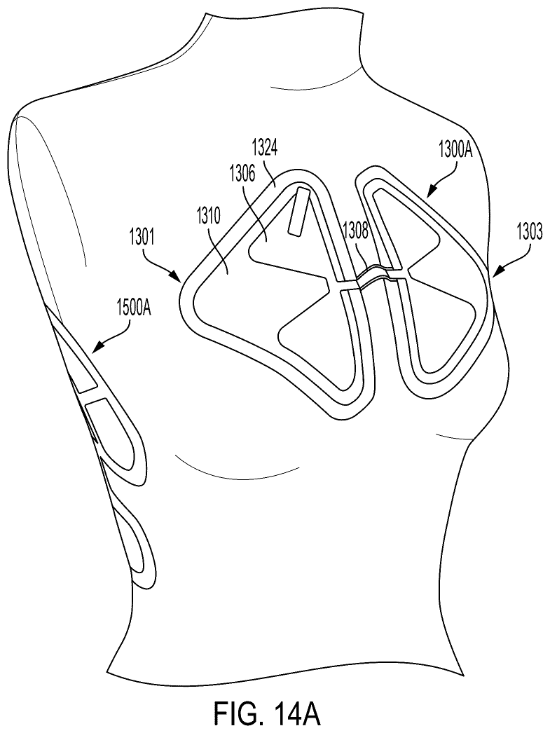

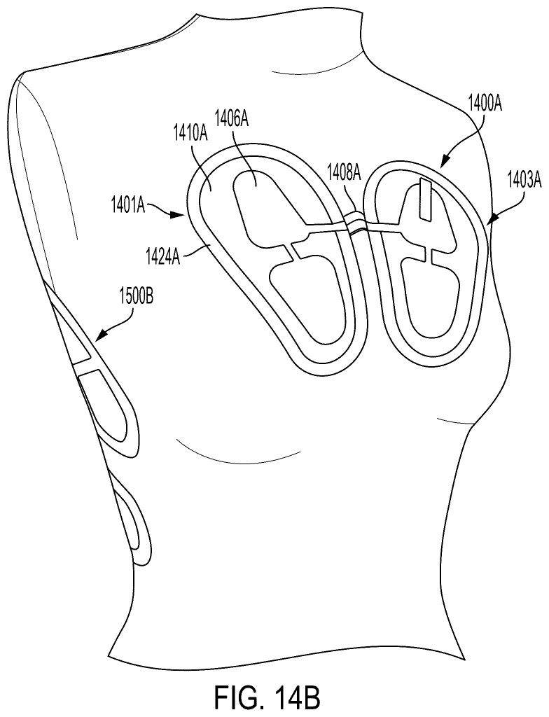

The flexible electrical connector 1308 may provide an adjustable distance between the first and the second electrode subassemblies 1301, 1303. The flexible electrical connector 1308 may be bent such that the first and the second electrode subassemblies 1301, 1303 are close together, but not touching (see, e.g., FIGS. 14A and 14B). When bent, the flexible electrical connector 1308 may form a tent-like shape where the apex points away from the subject, and thus, the flexible electrical connector 1308 may deform to a substantially concave shape away from the subject's body. The “tenting” of the flexible electrical connector 1308 may allow for improved heat dissipation. The flexible electrical connector 1308 may be pulled taught such that the first and the second electrode subassemblies 1301, 1303 are at a maximum distance, measured along the surface of the transducer apparatus 1300A.

The flexible electrical connector 1308 may have a length between the first and the second electrode subassemblies 1301, 1303 of at least 0.05 cm, or at least 0.1 cm, and not more than 5.00 cm. When viewed in a direction perpendicular to and toward the back face of the subassemblies 1301, 1303 (as in FIG. 13A), the flexible electrical connector 1308 may provide a distance S between the first and the second electrode subassemblies 1301, 1303. Due to the flexible electrical connector 1308 being flexible, the distance S between the first and the second electrode subassemblies 1301, 1303 may vary as the subject moves. The flexible electrical connector 1308 may provide a maximum distance S between the first and the second electrode subassemblies 1301, 1303 when the flexible electrical connector 1308 is planar.

In some embodiments, the distance S may be subject-specific. For example, the size of the subject may be considered when determining the subject-specific distance S, and an appropriately-sized transducer apparatus may be selected from a variety of sizes (e.g., small, medium, and large). Similarly, the width W of the flexible electrical connector 1308 is not particularly limited and can be selected in accordance with flexibility requirements.

The transducer apparatus 1300A may include one or more blank spaces 1326 (or void spaces), which do not overlap with any of the electrode elements 1302 and include only the anisotropic material layer 1310. Upon a rotational or translational shift of the subassembly, while (approximately) remaining in the preferred location with respect to treating a target in/on the subject's body, at least part of one or more of the blank spaces 1326 may be provide a relief region for the subject since no electrode elements 1302 are in the blank spaces 1326.

The transducer apparatus 1300A may further include a foam material layer 1324 directly or indirectly coupled to the anisotropic material layer 1310, located on a front face 1312 of the anisotropic material layer 1310, and configured to contact the subject's body. In some embodiments, a layer of conductive adhesive material or conductive hydrogel is located on the front face 1312 of the anisotropic material layer 1310 and may act as a biocompatible skin-contact layer. A front face 1348 of the foam material layer 1324 may include an adhesive to aid in securing the transducer apparatus 1300A to the subject's skin. When viewed in a direction perpendicular to and toward the front face of the subassemblies 1301, 1303 (as in FIG. 13B), the foam material layer 1324 may have an empty center such that the foam material layer 1324 frames the anisotropic material layer 1310. In some embodiments, the foam material layer 1324 may be a continuous layer that covers some or most of the front face 1312 of the anisotropic material layer 1310 but does not cover all of the front face 1312 of the anisotropic material layer 1310. In some embodiments, the anisotropic material layer 1310 may not extend outward all the way to the edge of the foam material layer 1324. The foam material layer 1324 may cover at most 5%, or at most 10%, or at most 20%, or at most 30%, or at most 40%, or at most 50%, or any percentages therebetween, of the anisotropic material layer 1310.

When viewed in a direction perpendicular to and toward the front face 1312 of the anisotropic material layer 1310 of each subassembly 1301, 1303, the foam material layer 1324 may include a perimeter 1346 covering a perimeter of the anisotropic material layer 1310. In some embodiments, the perimeter 1346 of the foam material layer 1324 may cover up to the edge of the perimeter of the anisotropic material layer 1310. In some embodiments, the perimeter 1346 of the foam material layer 1324 may be larger than the perimeter of the anisotropic material layer 1310. The perimeter 1346 of the foam material layer 1324 may have substantially the same shape as the perimeter of the anisotropic material layer 1310. In some embodiments, an offset distance P (not shown) between the perimeter 1346 of the foam material layer 1324 and the perimeter of the anisotropic material layer 1310 may be the same about the perimeter of the subassemblies 1301, 1303. Alternatively, the offset distance P between the perimeter of the anisotropic material layer 1310 and the perimeter portion 1346 of the foam material layer 1324 may vary about the perimeter of the subassemblies 1301, 1303.

The foam material layers 1334, 1336 may provide a comfortable surface against the subject's body. During certain treatments, the electrode subassemblies 1301, 1303 may need to be spaced apart at the maximum distance permitted by the length of the flexible electrical connector 1308. As such, the flexible electrical connector 1308 will be substantially flat and in contact or close to contact with the subject's body. The foam material layer 1334, 1336 may be included to provide a comfortable material in contact with the subject's body.

In some embodiments, the foam material layers 1324, 1334, and 1336 may be at least 2 mm and at most 10 mm thick, or at least 5 mm and at most 10 mm thick, or at least 8 mm and at most 10 mm thick. In some embodiments, the foam material layer 1324, 1334, 1336 may be formed at least partially of a soft material with airy open space. In some embodiments, the foam material layer 1324, 1334, 1336 may be formed of a non-porous material to improve sterilizability and cleanability. In some embodiments, the foam material layer 1324, 1334, 1336 may comprise one or more of low-density polyethylene (LDPE), silicone, polyurethane, or ethylene-vinyl acetate (EVA), each of which may be an open-cell, closed-cell, or partially open/closed-cell foam. In some embodiments, a closed-cell foam is preferred. In some embodiments, the foam may be an adhesive coated foam. In some embodiments, the foam may be a 3M™ Tegaderm™ product (3M, Saint Paul, MN, USA), which may include adhesive on one side facing the patient's body and facing away from the substrate 1304.

Each of the subassemblies 1301, 1303 may have separated portions of the substrate 1304. In some embodiments, the flexible electrical connector 1308 may not include a portion of the substrate 1304. In some embodiments, the substrate 1304 may not directly contact the subject's skin due to the foam material layer 1348. Suitable materials for the substrate 1304 may include, for example, tape, bandage, cloth, nonwoven fabric, foam, flexible plastic, and/or a conductive medical gel. The transducer 1300A may be affixed to the subject's body via the substrate 1304 (e.g., via an adhesive layer and/or a conductive medical gel). The substrate 1304 may be an adhesive bandage. The substrate may aid in securing a cable (not shown) connected to the flexible PCB 1306 for providing signals to the flexible PCB 1306 for generating TTFields with the transducer apparatus 1300A. As described above, in some embodiments, a skin-contact adhesive may be present on some or all of the anisotropic material layer 104, which may be any of those described above including a hydrogel or a biocompatible conductive adhesive or a non-adhesive conductive silicone elastomer as described herein.

FIGS. 14A and 14B depict a perspective view of an example transducer apparatus on a mannequin, according to some embodiments.

In FIG. 14A, the transducer apparatus 1300A of FIGS. 13A and 13B is depicted placed over the sternum area of a mannequin, and the target location (i.e. tumor location) for the TTFields therapy may be in the torso. For illustration purposes, the two electrode subassemblies 1301, 1303 are shown with the flexible PCB 1306, the anisotropic material layer 1310, and the foam layer 1324 and without the substrate 1304 (which would obscure these features). The two electrode subassemblies 1301, 1303 are spaced apart by the flexible electrical connector 1308. As placed on the mannequin, the flexible electrical connector 1308 includes a bend away from the subject and forms a substantially concave shape away from the subject. In some embodiments, when applying TTFields treatment, at least one pair of transducer apparatuses are employed. For example, as shown in FIG. 14A, the first transducer apparatus 1300A may be positioned on the subject's chest, and a second transducer apparatus of the first pair of transducer apparatuses may be positioned on the subject's back (not shown). Optionally, a second pair of transducer apparatuses may be positioned on the left and right sides of the subject's torso. For example, transducer apparatus 1500A, partly obscured, is shown located on one side of the subject's torso, which could be paired with a fourth transducer apparatus on the other side of the subject's torso (not shown).

In FIG. 14B, the transducer apparatus 1400A is depicted placed over the a sternum area of a mannequin, and the target location (i.e. tumor location) for the TTFields therapy may be in the torso. For illustration purposes, the two electrode subassemblies 1401A, 1403A are shown with the flexible PCB 1406A, the anisotropic material layer 1410A, and the foam layer 1424A and without the substrate 1404A (which would obscure these features). The two electrode subassemblies 1401A, 1403A are spaced apart by the flexible electrical connector 1408A. As placed on the mannequin, the flexible electrical connector 1408A includes a bend away from the subject and forms a substantially concave shape away from the subject. In some embodiments, when applying TTFields treatment, at least one pair of transducer apparatuses are employed. Transducer apparatus 1400A differs from transducer apparatus 1300A in illustrating different shapes for the electrode subassemblies 1401A, 1403A versus 1301A, 1303A, as well as different shapes for the flexible PCB 1406A (and, not visible, electrode elements 1402) compared to those of 1306A (and 1302) in transducer apparatus 1300A. As discussed above with respect to FIG. 14A, and similarly for FIG. 14B, the first transducer apparatus 1400A may be paired with a second transducer apparatus on the subject's back (not shown). Optionally, another pair of transducer apparatuses may be positioned on the left and right side of the subject's torso (one of which, 1500B, is partially visible in FIG. 14B).

### ILLUSTRATIVE EMBODIMENTS

The invention includes other illustrative embodiments (“Embodiments”) as follows.

Embodiment 1. A transducer apparatus for delivering tumor treating fields to a subject's body, the transducer apparatus comprising: a plurality of electrode subassemblies, each electrode subassembly configured to be positioned over the subject's body with a front face of the electrode subassembly facing the subject's body, each electrode subassembly having a back face opposite the front face, each electrode subassembly comprising at least one electrode element; a plurality of sections of an anisotropic material layer, each section of anisotropic material layer electrically coupled to at least one electrode element of at least one of the plurality of electrode subassemblies, each section of anisotropic material layer comprising a front face for facing the subject's body and a back face opposite the front face, the back face of each section of anisotropic material layer facing the at least one electrode element, and wherein when viewed from a direction perpendicular to the front face of the electrode subassemblies, each respective section of anisotropic material layer is spaced apart and is not touching other sections of the plurality of sections of anisotropic material layer; and a flexible electrical connector electrically connecting the plurality of electrode subassemblies.

Embodiment 2. The transducer apparatus of embodiment 1, wherein the plurality of sections of anisotropic material layer is equal in number to that of the plurality of electrode subassemblies, and each anisotropic material layer has an areal footprint that partially or wholly overlaps an areal footprint of each electrode subassembly, and, optionally, may extend laterally beyond the areal footprint of each electrode subassembly.

Embodiment 3. The transducer apparatus of embodiment 1, wherein one or more sections of the plurality of sections of anisotropic material layer has an areal footprint that partially or wholly overlaps an areal footprint of more than one electrode subassembly of the plurality of electrode subassemblies, and, optionally, may extend laterally beyond the areal footprint of the more than one electrode subassembly of the plurality of electrode subassemblies.

Embodiment 4. The transducer apparatus of embodiment 1, wherein when viewed from the direction perpendicular to the front face of the electrode subassemblies, each electrode subassembly comprises an areal footprint, and the areal footprint of the plurality of electrode subassemblies defines a substantially U-shape, a substantially V-shape, a substantially C-shape, or a substantially rounded V-shape.

Embodiment 5. The transducer apparatus of embodiment 1, wherein when viewed from the direction perpendicular to the front face of the electrode subassemblies, each electrode subassembly comprises an areal footprint, and the areal footprint of the plurality of electrode subassemblies defines a substantially circular shape or a substantially semi-circular shape.

Embodiment 6. The transducer apparatus of embodiment 1, wherein when viewed from the direction perpendicular to the front face of the electrode subassemblies, each electrode subassembly comprises an areal footprint, and the areal footprint of the plurality of electrode subassemblies defines a substantially triangular shape, a substantially quadrilateral shape, a substantially pentagonal shape, a substantially hexagonal shape, a substantially heptagonal shape, or a substantially octagonal shape.

Embodiment 7. The transducer apparatus of embodiment 1, further comprising a substrate having a front face and a back face opposite the front face, wherein the front face of the substrate is adjacent to the back face of each of the electrode subassemblies.

Embodiment 8. The transducer apparatus of embodiment 1, wherein each section of anisotropic material layer comprises a layer of graphite material, and wherein the layer is anisotropic with respect to at least one of thermal conductivity or electrical conductivity.

Embodiment 9. The transducer apparatus of embodiment 1, wherein the front face of each section of anisotropic material layer is in electrical contact with a first layer of conductive adhesive or conductive gel.

Embodiment 10. The transducer apparatus of embodiment 9, wherein the back face of each section of anisotropic material layer is in electrical contact with a second layer of conductive adhesive or conductive gel.

Embodiment 10A: The transducer apparatus of embodiment 10, wherein the first layer of conductive adhesive or gel comprises a first adhesive strength, the second layer of conductive adhesive or gel comprises a second adhesive strength, and, optionally, wherein the second adhesive strength is greater than the first adhesive strength.

Embodiment 10B. The transducer apparatus of Embodiment 1, wherein the front face of each section of the anisotropic material layer is in electrical contact with a respective layer of conductive silicone elastomer, wherein each layer of conductive silicone elastomer has a non-adhesive front face that is textured in a manner that makes it stick to human skin.

Embodiment 11. The transducer apparatus of embodiment 1, wherein each of the plurality of electrode subassemblies further comprises a printed circuit board or portion thereof disposed in electrical contact with the back face of a section of anisotropic material layer.

Embodiment 12. A transducer apparatus for delivering tumor treating fields to a subject's body, the transducer apparatus comprising: a plurality of electrode subassemblies, each electrode subassembly configured to be positioned over the subject's body with a front face of the electrode subassembly facing the subject's body, each electrode subassembly having a back face opposite the front face, each electrode subassembly comprising at least one electrode element; and an anisotropic material layer electrically coupled to the at least one electrode element, the anisotropic material layer comprising a front face facing the subject's body and a back face opposite the front face, the back face facing the at least one electrode element; wherein when viewed from a direction perpendicular to the front face of the electrode subassemblies, the anisotropic material layer is segmented into a plurality of sections of anisotropic material layer and the plurality of sections of anisotropic material layer define a substantially C-shape, a substantially U-shape, a substantially V-shape, a substantially rounded V-shape, or a substantially annular shape.

Embodiment 13. The transducer apparatus of embodiment 12, wherein when viewed from a direction perpendicular to the front face of the electrode subassemblies, the sections of anisotropic material layer are spaced apart and are not touching.

Embodiment 14. The transducer apparatus of embodiment 12, wherein when viewed from a direction perpendicular to the front face of the electrode subassemblies, each section of anisotropic material layer is connected to another section of anisotropic material layer with a thin bridging anisotropic material layer.

Embodiment 15. The transducer apparatus of embodiment 14, wherein when viewed from the direction perpendicular to the front face of the electrode subassemblies, the plurality of electrode subassemblies define a substantially U-shape, a substantially V-shape, a substantially C-shape, or a substantially rounded V-shape, wherein when viewed from the direction perpendicular to the front face of the electrode subassemblies, an interior edge of the substantially U-shape, the substantially V-shape, the substantially C-shape, or the substantially rounded V-shape defines a point of continuity for the thin bridging anisotropic material layers and adjoining sections of anisotropic material layer, and wherein each thin bridging anisotropic material layer has a width less than one third the width of a smallest width of its two adjoining sections of anisotropic material layer.

Embodiment 16. The transducer apparatus of embodiment 12, wherein when viewed from a direction perpendicular to the front face of the electrode subassemblies, the sections of anisotropic material layer define a contiguous shape with peninsula portions having an areal footprint sufficient to be coincident with one or more electrode subassembly.

Embodiment 17. The transducer apparatus of embodiment 12, wherein each of the plurality of electrode subassemblies further comprise a printed circuit board or portion thereof disposed in electrical contact with the back face of the respective anisotropic material layer.

Embodiment 18. The transducer apparatus of embodiment 12, wherein each of the plurality of electrode subassemblies is electrically connected to at least one other electrode subassembly via a flexible electrical connector.

Embodiment 18A: The transducer apparatus of embodiment 12, further comprising a substrate having a front face and a back face opposite the front face, wherein the front face of the substrate is adjacent the back face of each of the electrode subassemblies.

Embodiment 18B: The transducer apparatus of embodiment 12, wherein the anisotropic material layer comprises a layer of graphite material, and wherein the layer is anisotropic with respect to at least one of thermal conductivity or electrical conductivity.

Embodiment 18C: The transducer apparatus of embodiment 12, wherein the front face of the anisotropic material layer comprises a first layer of conductive adhesive or gel.

Embodiment 18D: The transducer apparatus of embodiment 17, wherein the back face of the anisotropic material layer comprises a second layer of conductive adhesive or gel.

Embodiment 18E: The transducer apparatus of embodiment 17, wherein the first layer of conductive adhesive or gel comprises a first adhesive strength, the second layer of conductive adhesive or gel comprises a second adhesive strength, and, optionally, wherein the second adhesive strength is greater than the first adhesive strength.

Embodiment 19. A transducer apparatus for delivering tumor treating fields to a subject's body, the transducer apparatus comprising: a plurality of electrode subassemblies, each electrode subassembly configured to be positioned over the subject's body with a front face of the electrode subassembly facing the subject's body, each subassembly having a back face opposite the front face, each electrode subassembly comprising at least one electrode element; and a plurality of sections of an anisotropic material layer, each section electrically coupled to at least one electrode element of at least one of the plurality of electrode subassemblies, each section of anisotropic material layer comprising a front face facing the subject's body and a back face opposite the front face, the back face facing the at least one electrode element; wherein when viewed from a direction perpendicular to the front face of the electrode subassemblies, the sections of anisotropic material layer are not contiguous, wherein when viewed from the direction perpendicular to the front face of the electrode subassemblies, each of the sections of anisotropic material layer define a same shape.

Embodiment 20. The transducer apparatus of embodiment 19, wherein the plurality of electrode subassemblies define a centroid, and the plurality of electrode subassemblies are rotationally symmetric about the centroid.

Embodiment 21. The transducer apparatus of embodiment 19, wherein each of the electrode subassemblies defines a circular shape.

Embodiment 22. The transducer apparatus of embodiment 19, wherein each of the electrode subassemblies defines a teardrop or petal shape.

Embodiment 23. The transducer apparatus of embodiment 19, wherein each electrode subassembly comprises: one electrode element; and an anisotropic material layer, wherein when viewed from a direction perpendicular to the front face of the subassembly, the anisotropic material layer has an areal footprint having a size and shape that substantially matches that of the electrode element.

Embodiment A1. A transducer apparatus for delivering tumor treating fields to a subject's body, the transducer apparatus comprising: a plurality of electrode subassemblies, each subassembly configured to be positioned over the subject's body with a front face of the subassembly facing the subject's body, each subassembly having a back face opposite the front face, each subassembly comprising at least one electrode element; and a flexible electrical connector electrically connecting the plurality of electrode subassemblies, wherein the plurality of electrode subassemblies are spaced away from each other and movable relative to one another.

Embodiment A2. The transducer apparatus of Embodiment A1, wherein each electrode subassembly further comprises an adhesive layer.

Embodiment A3. The transducer apparatus of Embodiment A2, wherein the adhesive layer is disposed on the front faces of the plurality of electrode subassemblies, respectively.

Embodiment A4. The transducer apparatus of Embodiment A3, wherein the adhesive layer comprises a mechanical adhesive.

Embodiment A5. The transducer apparatus of Embodiment A2, wherein the adhesive layer is disposed on the back faces of the plurality of electrode subassemblies, respectively.

Embodiment A6. The transducer apparatus of Embodiment A5, wherein the adhesive layer comprises a flexible substrate.

Embodiment A7. The transducer apparatus of Embodiment A1, wherein the plurality of electrode subassemblies each comprise an anisotropic material layer which is electrically coupled to the at least one electrode element, the anisotropic material layer comprising a front face facing the subject's body and a back face opposite the front face, the back face facing the at least one first electrode element, wherein the anisotropic material layer of the first electrode subassembly is distinct from the anisotropic material layer of the second electrode subassembly.

Embodiment A7A. The transducer apparatus of Embodiment A7, wherein the front face of each section of the anisotropic material layer is in electrical contact with a respective layer of conductive silicone elastomer, wherein each layer of conductive silicone elastomer has a non-adhesive front face that is textured in a manner that makes it stick to human skin.

Embodiment A8. The transducer apparatus of Embodiment A1, wherein a space between each adjacent electrode subassembly of the plurality of electrode subassemblies is greater than or equal to 1 mm and less than or equal to 10 mm.

Embodiment A9. The transducer apparatus of Embodiment A1, wherein the plurality of electrode subassemblies collectively form an electrode superassembly, and wherein the electrode superassembly comprises a flexible printed circuit board.

Embodiment A10. The transducer apparatus of Embodiment A9, wherein the electrode superassembly defines a substantially M-shape, a substantially N-shape, a substantially W-shape, a substantially O-shape, a substantially U-shape, a substantially V-shape, a substantially C-shape, or a substantially D-shape.

Embodiment A11. The transducer apparatus of Embodiment A1, wherein the plurality of electrode subassemblies comprises at least two electrode subassemblies and at most thirteen electrode subassemblies.

Embodiment A11A. The transducer apparatus of Embodiment A1, wherein the flexible electrical connector comprises a fluoropolymer wrapper.

Embodiment A12. A transducer apparatus for delivering tumor treating fields to a subject's body, the transducer apparatus comprising: a plurality of electrode subassemblies, each subassembly configured to be positioned over the subject's body with a front face of the subassembly facing the subject's body, each subassembly having a back face opposite the front face, each subassembly comprising: at least one electrode element; an anisotropic material layer electrically coupled to the at least one electrode element, the anisotropic material layer comprising a front face facing the subject's body and a back face opposite the front face, the back face facing the at least one first electrode element; and a flexible electrical connector electrically connecting the plurality of electrode subassemblies; wherein the plurality of electrode subassemblies are spaced away from each other and movable relative to one another; and wherein when viewed from a direction perpendicular to the front face of the electrode subassemblies, the anisotropic material layer is segmented into a plurality of sections of anisotropic material layer and the plurality of sections of anisotropic material layer define a substantially C-shape, a substantially U-shape, a substantially V-shape, a substantially rounded V-shape, or a substantially annular shape.

Embodiment A13. The transducer apparatus of Embodiment A12, further comprising a conductive adhesive layer located on the front face of the anisotropic material layer.

Embodiment A14. The transducer apparatus of Embodiment A13, wherein the conductive adhesive layer comprises a conductive medical gel, a hydrogel, or a conductive silicone elastomer.

Embodiment A15. The transducer apparatus of Embodiment A13, wherein the conductive adhesive layer is a mechanical adhesive.

Embodiment A16. The transducer apparatus of Embodiment A12, wherein the anisotropic material layer comprises a layer of graphite material, and wherein the layer is anisotropic with respect to at least one of thermal conductivity or electrical conductivity.

Embodiment A17. The transducer apparatus of Embodiment A12, a space between each adjacent electrode subassembly of the plurality of electrode subassemblies is greater than or equal to 1 mm and less than or equal to 10 mm.

Embodiment A18. A transducer apparatus for delivering tumor treating fields to a subject's body, the transducer apparatus comprising: plurality of electrode subassemblies, each subassembly configured to be positioned over the subject's body with a front face of the subassembly facing the subject's body, each subassembly having a back face opposite the front face, each subassembly comprising: at least one electrode element; a substrate comprising a front face facing the subject's body and a back face opposite the front face, the front face of the substrate facing the first and second electrode subassemblies, and a flexible electrical connector electrically connecting the plurality of electrode subassemblies; wherein the plurality of electrode subassemblies are spaced away from each other and movable relative to one another; and wherein when viewed from a direction perpendicular to the front face of the electrode subassemblies, the substrate is segmented into a plurality of sections.

Embodiment A19. The transducer apparatus of Embodiment A18, wherein each section of the substrate comprises an outer perimeter and, when viewed from a direction perpendicular to the front faces of the plurality of electrode subassemblies, each of the plurality of electrode subassemblies is positioned within the outer perimeter of the respective section of substrate.

Embodiment A19A. The transducer apparatus of Embodiment A18, when viewed from the direction perpendicular to the front face of the electrode subassemblies, the sections of the substrate define a substantially C-shape, a substantially U-shape, a substantially V-shape, a substantially rounded V-shape, or a substantially annular shape.

Embodiment A20. The transducer apparatus of Embodiment A18, wherein at least a part of the front face of the substrate comprises an adhesive layer.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
a plurality of electrode subassemblies, each electrode subassembly configured to be positioned over the subject's body with a front face of the electrode subassembly facing the subject's body, each electrode subassembly having a back face opposite the front face, each electrode subassembly comprising at least one electrode element;
a plurality of sections of an anisotropic material layer, each section of anisotropic material layer electrically coupled to at least one electrode element of at least one of the plurality of electrode subassemblies, each section of anisotropic material layer comprising a front face for facing the subject's body and a back face opposite the front face, the back face of each section of anisotropic material layer facing the at least one electrode element, and wherein when viewed from a direction perpendicular to the front face of the electrode subassemblies, each respective section of anisotropic material layer is spaced apart and is not touching other sections of the plurality of sections of anisotropic material layer; and
a flexible electrical connector electrically connecting the plurality of electrode subassemblies.

2. The transducer apparatus of claim 1, wherein the plurality of sections of anisotropic material layer is equal in number to that of the plurality of electrode subassemblies, and each anisotropic material layer has an areal footprint that partially or wholly overlaps an areal footprint of each electrode subassembly, and, optionally, may extend laterally beyond the areal footprint of each electrode subassembly.

3. The transducer apparatus of claim 1, wherein one or more sections of the plurality of sections of anisotropic material layer has an areal footprint that partially or wholly overlaps an areal footprint of more than one electrode subassembly of the plurality of electrode subassemblies, and, optionally, may extend laterally beyond the areal footprint of the more than one electrode subassembly of the plurality of electrode subassemblies.

4. The transducer apparatus of claim 1, wherein when viewed from the direction perpendicular to the front face of the electrode subassemblies, each electrode subassembly comprises an areal footprint, and the areal footprint of the plurality of electrode subassemblies defines a substantially U-shape, a substantially V-shape, a substantially C-shape, or a substantially rounded V-shape.

5. The transducer apparatus of claim 1, further comprising a substrate having a front face and a back face opposite the front face, wherein the front face of the substrate is adjacent to the back face of each of the electrode subassemblies.

6. The transducer apparatus of claim 1, wherein each section of anisotropic material layer comprises a layer of graphite material, and wherein the layer is anisotropic with respect to at least one of thermal conductivity or electrical conductivity.

7. The transducer apparatus of claim 1, wherein the front face of each section of anisotropic material layer is in electrical contact with a first layer of conductive adhesive or conductive gel.

8. The transducer apparatus of claim 7, wherein the back face of each section of anisotropic material layer is in electrical contact with a second layer of conductive adhesive or conductive gel.

9. The transducer apparatus of claim 1, wherein the front face of each section of the anisotropic material layer is in electrical contact with a respective layer of conductive silicone elastomer, wherein each layer of conductive silicone elastomer has a non-adhesive front face that is textured in a manner that makes it stick to human skin.

10. The transducer apparatus of claim 1, wherein each of the plurality of electrode subassemblies further comprises a printed circuit board or portion thereof disposed in electrical contact with the back face of a section of anisotropic material layer.

11. A transducer apparatus for delivering tumor treating fields to a subject's body, the transducer apparatus comprising:
a plurality of electrode subassemblies, each electrode subassembly configured to be positioned over the subject's body with a front face of the electrode subassembly facing the subject's body, each electrode subassembly having a back face opposite the front face, each electrode subassembly comprising at least one electrode element; and
an anisotropic material layer electrically coupled to the at least one electrode element, the anisotropic material layer comprising a front face facing the subject's body and a back face opposite the front face, the back face facing the at least one electrode element;
wherein when viewed from a direction perpendicular to the front face of the electrode subassemblies, the anisotropic material layer is segmented into a plurality of sections of anisotropic material layer and the plurality of sections of anisotropic material layer define a substantially C-shape, a substantially U-shape, a substantially V-shape, a substantially rounded V-shape, or a substantially annular shape.

12. The transducer apparatus of claim 11, wherein when viewed from a direction perpendicular to the front face of the electrode subassemblies, the sections of anisotropic material layer are spaced apart and are not touching.

13. The transducer apparatus of claim 11, wherein when viewed from a direction perpendicular to the front face of the electrode subassemblies, each section of anisotropic material layer is connected to another section of anisotropic material layer with a thin bridging anisotropic material layer.

14. The transducer apparatus of claim 13, wherein when viewed from the direction perpendicular to the front face of the electrode subassemblies, the plurality of electrode subassemblies define a substantially U-shape, a substantially V-shape, a substantially C-shape, or a substantially rounded V-shape,
wherein when viewed from the direction perpendicular to the front face of the electrode subassemblies, an interior edge of the substantially U-shape, the substantially V-shape, the substantially C-shape, or the substantially rounded V-shape defines a point of continuity for the thin bridging anisotropic material layers and adjoining sections of anisotropic material layer, and wherein each thin bridging anisotropic material layer has a width less than one third the width of a smallest width of its two adjoining sections of anisotropic material layer.

15. The transducer apparatus of claim 11, wherein each of the plurality of electrode subassemblies further comprise a printed circuit board or portion thereof disposed in electrical contact with the back face of the respective anisotropic material layer.

16. A transducer apparatus for delivering tumor treating fields to a subject's body, the transducer apparatus comprising:
a plurality of electrode subassemblies, each electrode subassembly configured to be positioned over the subject's body with a front face of the electrode subassembly facing the subject's body, each subassembly having a back face opposite the front face, each electrode subassembly comprising at least one electrode element; and
a plurality of sections of an anisotropic material layer, each section electrically coupled to at least one electrode element of at least one of the plurality of electrode subassemblies, each section of anisotropic material layer comprising a front face facing the subject's body and a back face opposite the front face, the back face facing the at least one electrode element;
wherein when viewed from a direction perpendicular to the front face of the electrode subassemblies, the sections of anisotropic material layer are not contiguous,
wherein when viewed from the direction perpendicular to the front face of the electrode subassemblies, each of the sections of anisotropic material layer define a same shape.

17. The transducer apparatus of claim 16, wherein the plurality of electrode subassemblies define a centroid, and the plurality of electrode subassemblies are rotationally symmetric about the centroid.

18. The transducer apparatus of claim 16, wherein each of the electrode subassemblies defines a circular shape.

19. The transducer apparatus of claim 16, wherein each of the electrode subassemblies defines a teardrop or petal shape.

20. The transducer apparatus of claim 16, wherein each electrode subassembly comprises:
one electrode element; and
an anisotropic material layer, wherein when viewed from a direction perpendicular to the front face of the subassembly, the anisotropic material layer has an areal footprint having a size and shape that substantially matches that of the electrode element.
